(19) United States
(12) Patent Application Publication
(10) Pub. No.: US 2020/0085838 A1
(43) Pub. Date: Mar. 19, 2020

(54) METHODS OF TREATING EPILEPSY AND NEURODEVELOPMENTAL DISORDERS

(71) Applicant: Praxis Precision Medicines, Inc., Cambridge, MA (US)

(72) Inventors: Gabriel Martinez Botella, Wayland, MA (US); Kiran Reddy, Boston, MA (US); Marion Wittmann, Medford, MA (US)

(21) Appl. No.: 16/613,994

(22) PCT Filed: May 16, 2018
(86) PCT No.: PCT/US2018/033028

§ 371 Date: Nov. 15, 2019

Related U.S. Application Data
(60) Provisional application No. 62/507,145, filed on May 16, 2017.
(60) Provisional application No. 62/507,301, filed on May 17, 2017.

(51) Int. Cl. A61K 31/553; A61P 25/08

(52) U.S. Cl. CPC A61K 31/553; A61P 25/08

(57) ABSTRACT

The present invention is directed to, in part, methods of preventing and/or treating a disease or condition relating to aberrant function of a voltage-gated, sodium ion channel, for example, abnormal late/persistent sodium current. In some embodiments, methods of treating a neurodevelopmental disorder (e.g., epilepsy) comprising administering to a subject in need thereof a compound disclosed herein (e.g., a compound of Formula (I), (I-a), or (I-b), e.g., eleclazine) are provided.

### CROSS REFERENCE TO RELATED APPLICATIONS

This application claims priority to and the benefit of U.S. Provisional Patent Application No. 62/507,145, filed May 16, 2017, and U.S. Provisional Patent Application No. 62/507,301, filed May 17, 2017, each of which is incorporated herein by reference in its entirety.

### BACKGROUND

Sodium ion (Na+) channels primarily open in a transient manner and are quickly inactivated, thereby generating a fast Na+ current to initiate the action potential. The late or persistent sodium current (INaL) is a sustained component of the fast Na+ current of cardiac myocytes and neurons. Many common neurological and cardiac conditions are associated with abnormal INaL enhancement, which contributes to the pathogenesis of both electrical and contractile dysfunction in mammals (see, e.g., Pharmacol Ther (2008) 119:326-339). Accordingly, pharmaceutical compounds that selectively modulate sodium channel activity, e.g., abnormal INaL, are useful in treating such disease states.

### SUMMARY OF THE INVENTION

Described herein are methods of treating and diagnosing a subject. In an embodiment, the subject has an epilepsy characterized by a mutation in an epilepsy associated gene or autism associated gene. In an embodiment, the subject is characterized as having normal cardiac function.

In one aspect, the invention features a method for treating a subject having an epilepsy, wherein the epilepsy is characterized by a mutation in an epilepsy associated gene or autism associated gene, the method comprising administering to the subject a modulator (e.g., an inhibitor) of a sodium ion channel to treat the epilepsy.

In an embodiment, the mutation is a spontaneous mutation. In an embodiment, the mutation is determined in the subject by sequencing of the DNA or RNA from a sample of the subject. In an embodiment, the sequencing comprises sequencing of the whole genome or whole exome of the subject. In an embodiment, the sequencing comprises sequencing a panel of genes from the subject. In an embodiment, the mutation is a mutation in an epilepsy associated gene. In an embodiment, the mutation is a mutation identified in the Epilepsy phenome/Genome project. In an embodiment, the mutation is a mutation in the gene selected from ALDH7A1, ALG13, ARHGEF9, ARX, ASAH1, CDKL5, CHD2, CHRNA2, CHRNA4, CHRNB2, CLN8, CNTNAP2, CPA6, CSTB, DEPDC5, DNM1, EEF1A2, EPM2A, EPM2B, GABRA1, GABRB3, GABRG2, GNAO1, GOSR2, GRIN1, GRIN2A, GRIN2B, HCN1, IER3IP1, KCNA2, KCNB1, KCNC1, KCNMA1, KCNQ2, KCNQ3, KCNT1, KCTD7, LGI1, MEF2C, NHLRC1, PCDH19, PLCB1, PNKP, PNPO, PRICKLE1, PRICKLE2, PRRT2, RELN, SCARB2, SCN1A, SCN1B, SCN2A, SCN8A, SCN9A, SIAT9, SIK1, SLC13A5, SLC25A22, SLC2A1, SLC35A2, SLC6A1, SNIP1, SPTAN1, SRPX2, ST3GAL3, STRADA, STX1B, STXBP1, SYN1, SYNGAP1, SZT2, TBC1D24, and WWOX. In an embodiment, the mutation is in an autism associated gene. In an embodiment, the modulator is a modulator (e.g., an inhibitor) of late sodium current (INaL). In an embodiment, the modulator of the sodium channel is eleclazine.

In another aspect, the invention provides a method of treating a neurodevelopmental disorder, wherein the method comprises administering to a subject in need thereof a compound of Formula (I-a):

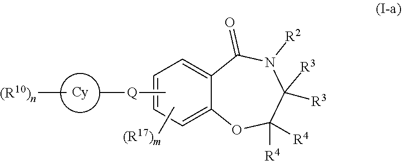

or a pharmaceutically acceptable salt, ester, stereoisomer, mixture of stereoisomers or tautomer thereof, wherein the variables are as defined herein.

In another aspect, the present invention provides a method of treating a pediatric epilepsy, wherein the method comprises administering to a subject in need thereof a compound of Formula (I-a):

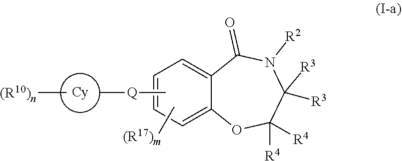

or a pharmaceutically acceptable salt, ester, stereoisomer, mixture of stereoisomers or tautomer thereof, wherein the variables are as defined herein.

In another aspect, the present invention provides a method of treating a refractory epilepsy, wherein the method comprises administering to a subject in need thereof a compound of Formula (I-a):

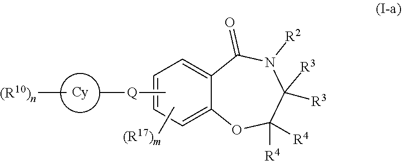

or a pharmaceutically acceptable salt, ester, stereoisomer, mixture of stereoisomers or tautomer thereof, wherein the variables are as defined herein.

In another aspect, the invention features, a method of diagnosing and treating a subject suffering from a epilepsy, the method comprising:
a. diagnosing whether the subject has an epilepsy that is susceptible to a treatment, the treatment comprising administration of a modulator (e.g., an inhibitor) of sodium channel, based on the presence of a mutation in an epilepsy associated gene or autism associated gene previously determined to be present in a sample from the subject; and
b. administering to the subject the treatment in an amount effective to treat the epilepsy.

In an embodiment, the mutation is a spontaneous mutation. In an embodiment, the mutation is determined in the subject by sequencing of the DNA or RNA from a sample of the subject. In an embodiment, the sequencing comprises sequencing of the whole genome or whole exome of the subject. In an embodiment, the sequencing comprises sequencing a panel of genes from the subject. In an embodiment, the mutation is a mutation in an epilepsy associated gene. In an embodiment, the mutation is a mutation identified in the Epilepsy phenome/Genome project. In an embodiment, the mutation is a mutation in the gene selected from ALDH7A1, ALG13, ARHGEF9, ARX, ASAH1, CDKL5, CHD2, CHRNA2, CHRNA4, CHRNB2, CLN8, CNTNAP2, CPA6, CSTB, DEPDC5, DNM1, EEF1A2, EPM2A, EPM2B, GABRA1, GABRB3, GABRG2, GNAO1, GOSR2, GRIN1, GRIN2A, GRIN2B, HCN1, IER3IP1, KCNA2, KCNB1, KCNC1, KCNMA1, KCNQ2, KCNQ3, KCNT1, KCTD7, LGI1, MEF2C, NHLRC1, PCDH19, PLCB1, PNKP, PNPO, PRICKLE1, PRICKLE2, PRRT2, RELN, SCARB2, SCN1A, SCN1B, SCN2A, SCN8A, SCN9A, SIAT9, SIK1, SLC13A5, SLC25A22, SLC2A1, SLC35A2, SLC6A1, SNIP1, SPTAN1, SRPX2, ST3GAL3, STRADA, STX1B, STXBP1, SYN1, SYNGAP1, SZT2, TBC1D24, and WWOX. In an embodiment, the mutation is in an autism associated gene. In an embodiment, the inhibitor is an inhibitor of late sodium current (INaL). In an embodiment, the inhibitor of a sodium channel is eleclazine.

In another aspect, the invention features a method for treating a subject having an epilepsy, wherein the subject is characterized as having normal cardiac function, the method comprising administering to the subject a modulator (e.g., an inhibitor) of a sodium ion channel to treat the epilepsy. In an embodiment, the modulator is a modulator (e.g., an inhibitor) of late sodium current (INaL). In an embodiment, the modulator of the sodium channel is eleclazine. In an embodiment, the subject is characterized as being without structural heart disease. In an embodiment, the subject is characterized as being without arrhythmias. In an embodiment, the method further comprises receiving information characterizing the subject based on an electrocardiogram. In an embodiment, the method further comprises receiving information characterizing the subject based on a cardiac ultrasound. In an embodiment, the method further comprises receiving information characterizing the subject based on an echocardiogram (e.g., relating to structural heart disease or arrhythmia). In an embodiment, the method further comprises receiving information characterizing the subject based on a cardiac MRI. In an embodiment, the method further comprises receiving information characterizing the subject based on a cardiac CT.

Other objects and advantages will become apparent to those skilled in the art from a consideration of the ensuing Detailed Description, Examples, and Claims.

### DETAILED DESCRIPTION OF THE INVENTION

As generally described herein, the present invention provides methods of preventing and/or treating a disease, disorder, or condition described herein, e.g., a disease, disorder, or condition relating to aberrant function of a sodium ion channel, such as abnormal late sodium current (INaL). In an embodiment, the epilepsy is characterized by a mutation in an epilepsy associated gene or autism associated gene. In an embodiment, the subject is characterized as having normal cardiac function. Also described herein are methods of diagnosing a subject for treatment with a modulator (e.g., an inhibitor) of a sodium ion channel (e.g., late sodium current (INaL)). Exemplary diseases, disorders, or conditions include epilepsy or an epilepsy syndrome, a neurodevelopmental disorder, pain, migraine, headache, a neuromuscular disorder, or a psychiatric disorder (e.g., bipolar disorder).

### Definitions

A “subject” to which administration is contemplated includes, but is not limited to, humans (i.e., a male or female of any age group, e.g., a pediatric subject (e.g, infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. The terms “human,” “patient,” and “subject” are used interchangeably herein.

Disease, disorder, and condition are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (“therapeutic treatment”), and also contemplates an action that occurs before a subject begins to suffer from the specified disease, disorder or condition (“prophylactic treatment”).

In general, the “effective amount” of a compound refers to an amount sufficient to elicit the desired biological response. As will be appreciated by those of ordinary skill in this art, the effective amount of a compound of the invention may vary depending on such factors as the desired biological endpoint, the pharmacokinetics of the compound, the disease being treated, the mode of administration, and the age, health, and condition of the subject. An effective amount encompasses therapeutic and prophylactic treatment.

As used herein, and unless otherwise specified, a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

As used herein, and unless otherwise specified, a “prophylactically effective amount” of a compound is an amount sufficient to prevent a disease, disorder or condition, or one or more symptoms associated with the disease, disorder or condition, or prevent its recurrence. A prophylactically effective amount of a compound means an amount of a therapeutic agent, alone or in combination with other agents, which provides a prophylactic benefit in the prevention of the disease, disorder or condition. The term “prophylactically effective amount” can encompass an amount that improves overall prophylaxis or enhances the prophylactic efficacy of another prophylactic agent.

### Methods of Treatment

Described herein are compounds and compositions thereof and their use to treat a disease, disorder, or condition relating to aberrant function of a sodium channel ion channel, e.g., abnormal late sodium (INaL) current. In some embodiments, a compound provided by the present invention is effective in the treatment of epilepsy or an epilepsy syndrome, a neurodevelopmental disorder, pain, or a neuromuscular disorder. Compounds of the invention may also modulate all sodium ion channels, or may be specific to only one or a plurality of sodium ion channels, e.g., Nay 1.1, 1.2, 1.5, 1.6, 1.7, 1.8, and/or 1.9.

In typical embodiments, the present invention is intended to encompass the compounds disclosed herein, and the pharmaceutically acceptable salts, pharmaceutically acceptable esters, tautomeric forms, polymorphs, and prodrugs of such compounds. In some embodiments, the present invention includes a pharmaceutically acceptable addition salt, a pharmaceutically acceptable ester, a hydrate of an addition salt, a tautomeric form, a polymorph, an enantiomer, a mixture of enantiomers, a stereoisomer or mixture of stereoisomers (pure or as a racemic or non-racemic mixture) of a compound described herein (e.g., a compound of Formula (I), (I-a), or (I-b), e.g., eleclazine).

### Epilepsy and Epilepsy Syndromes

The compounds described herein are useful in the treatment of epilepsy and epilepsy syndromes. Epilepsy is a CNS disorder in which nerve cell activity in the brain becomes disrupted, causing seizures or periods of unusual behavior, sensations and sometimes loss of consciousness. Seizure symptoms will vary widely, from a simple blank stare for a few seconds to repeated twitching of their arms or legs during a seizure.

Epilepsy may involve a generalized seizure or a partial or focal seizure. All areas of the brain are involved in a generalized seizure. A person experiencing a generalized seizure may cry out or make some sound, stiffen for several seconds to a minute a then have rhythmic movements of the arms and legs. The eyes are generally open, the person may appear not to be breathing and actually turn blue. The return to consciousness is gradual and the person maybe confused from minutes to hours. There are six main types of generalized seizures: tonic-clonic, tonic, clonic, myoclonic, absence, and atonic seizures. In a partial or focal seizure, only part of the brain is involved, so only part of the body is affected. Depending on the part of the brain having abnormal electrical activity, symptoms may vary.

Epilepsy, as described herein, includes a generalized, partial, complex partial, tonic clonic, clonic, tonic, refractory seizures, status epilepticus, absence seizures, febrile seizures, or temporal lobe epilepsy.

The compounds described herein may also be useful in the treatment of epilepsy syndromes. Severe syndromes with diffuse brain dysfunction caused, at least partly, by some aspect of epilepsy, are also referred to as epileptic encephalopathies. These are associated with frequent seizures that are resistant to treatment and severe cognitive dysfunction, for instance West syndrome.

In some embodiments, the epilepsy syndrome comprises an epileptic encephalopathy, such as Dravet syndrome, Angelman syndrome, CDKL5 disorder, frontal lobe epilepsy, infantile spasms, West's syndrome, Juvenile Myoclonic Epilepsy, Landau-Kleffner syndrome, Lennox-Gastaut syndrome, Ohtahara syndrome, PCDH19 epilepsy, or Glut1 deficiency.

In some embodiments, the epilepsy or epilepsy syndrome is a genetic epilepsy or a genetic epilepsy syndrome. In some embodiments, epilepsy or an epilepsy syndrome comprises epileptic encephalopathy, epileptic encephalopathy with SCN1A, SCN2A, SCN8A mutations, early infantile epileptic encephalopathy, Dravet syndrome, Dravet syndrome with SCN1A mutation, generalized epilepsy with febrile seizures, intractable childhood epilepsy with generalized tonic-clonic seizures, infantile spasms, benign familial neonatal-infantile seizures, SCN2A epileptic encephalopathy, focal epilepsy with SCN3A mutation, cryptogenic pediatric partial epilepsy with SCN3A mutation, SCN8A epileptic encephalopathy, sudden unexpected death in epilepsy, Rasmussen encephalitis, malignant migrating partial seizures of infancy, autosomal dominant nocturnal frontal lobe epilepsy, sudden expected death in epilepsy (SUDEP), KCNQ2 epileptic encephalopathy, or KCNT1 epileptic encephalopathy.

In some embodiments, the methods described herein further comprise identifying a subject having epilepsy or an epilepsy syndrome (e.g., epileptic encephalopathy, epileptic encephalopathy with SCN1A, SCN2A, SCN8A mutations, early infantile epileptic encephalopathy, Dravet syndrome, Dravet syndrome with SCN1A mutation, generalized Epilepsy with febrile seizures, intractable childhood epilepsy with generalized tonic-clonic seizures, infantile spasms, benign familial neonatal-infantile seizures, SCN2A epileptic encephalopathy, focal epilepsy with SCN3A mutation, cryptogenic pediatric partial epilepsy with SCN3A mutation, SCN8A epileptic encephalopathy, sudden unexpected death in epilepsy, Rasmussen encephalitis, malignant migrating partial seizures of infancy, autosomal dominant nocturnal frontal lobe epilepsy, sudden expected death in epilepsy (SUDEP), KCNQ2 epileptic encephalopathy, or KCNT1 epileptic encephalopathy) prior to administration of a compound described herein (e.g., a compound of Formula (I), (I-a), or (I-b), e.g., eleclazine).

In one aspect, the present invention features a method of treating epilepsy or an epilepsy syndrome (e.g., epileptic encephalopathy, epileptic encephalopathy with SCN1A, SCN2A, SCN8A mutations, early infantile epileptic encephalopathy, Dravet syndrome, Dravet syndrome with SCN1A mutation, generalized Epilepsy with febrile seizures, intractable childhood epilepsy with generalized tonic-clonic seizures, infantile spasms, benign familial neonatal-infantile seizures, SCN2A epileptic encephalopathy, focal epilepsy with SCN3A mutation, cryptogenic pediatric partial epilepsy with SCN3A mutation, SCN8A epileptic encephalopathy, sudden unexpected death in epilepsy, Rasmussen encephalitis, malignant migrating partial seizures of infancy, autosomal dominant nocturnal frontal lobe epilepsy, sudden expected death in epilepsy (SUDEP), KCNQ2 epileptic encephalopathy, or KCNT1 epileptic encephalopathy) comprising administering to a subject in need thereof a compound of Formula (I):

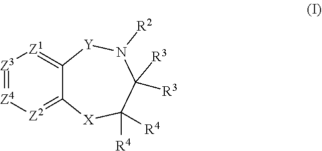

or a pharmaceutically acceptable salt thereof, wherein X, Y, Z¹, Z², Z³, Z⁴, R², R³, R⁴ and subvariables thereof are as defined herein.

In some embodiments, Z¹ is CR⁷ (e.g., CH). In some embodiments, Z² is CR⁷ (e.g., CH). In some embodiments, each of Z¹ and Z² is independently CR⁷ (e.g., CH).

In some embodiments, Z³ is C-Q-R¹. In some embodiments, Q is a covalent bond. In some embodiments, R¹ is aryl (e.g., phenyl) optionally substituted with —O—R²⁰ (e.g., —OCF₃). In some embodiments, Z⁴ is CR⁷ (e.g., CH).

In some embodiments, Y is —C(O)—. In some embodiments, X is —O—.

In some embodiments, R² is —C₁₋₆ alkylene-R⁵. In some embodiments, R² is —CH₂—R⁵. In some embodiments, R⁵ is heteroaryl (e.g., a nitrogen-containing heteroaryl, e.g., pyrimidinyl).

In some embodiments, each R³ is independently hydrogen. In some embodiments, each R⁴ is independently hydrogen. In some embodiments, each R³ and R⁴ is independently hydrogen.

In some embodiments, the compound of Formula (I) is 4-(pyrimidin-2-ylmethyl)-7-(4-(trifluoromethoxy)phenyl)-3,4-dihydrobenzo[f][1,4]oxazepin-5(2H)-one (i.e., eleclazine, GS-6615, CAS 1443211-72-0), or a pharmaceutically acceptable salt, ester, stereoisomer, mixture of stereoisomers or tautomer thereof. In some embodiments, the compound of Formula (I) is

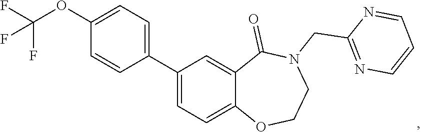 [SMILES: O=C1c2cc(-c3ccc(OC(F)(F)F)cc3)ccc2OCCN1Cc1ncccn1]

or a pharmaceutically acceptable salt, ester, stereoisomer, mixture of stereoisomers or tautomer thereof.

A compound of the present invention (e.g., a compound of Formula (I), (I-a), or (I-b), e.g., eleclazine) may also be used to treat an epileptic encephalopathy, wherein the subject has a mutation in one or more of ALDH7A1, ALG13, ARHGEF9, ARX, ASAH1, CDKL5, CHD2, CHRNA2, CHRNA4, CHRNB2, CLN8, CNTNAP2, CPA6, CSTB, DEPDC5, DNM1, EEF1A2, EPM2A, EPM2B, GABRA1, GABRB3, GABRG2, GNAO1, GOSR2, GRIN1, GRIN2A, GRIN2B, HCN1, IER3IP1, KCNA2, KCNB1, KCNC1, KCNMA1, KCNQ2, KCNQ3, KCNT1, KCTD7, LGI1, MEF2C, NHLRC1, PCDH19, PLCB1, PNKP, PNPO, PRICKLE1, PRICKLE2, PRRT2, RELN, SCARB2, SCN1A, SCN1B, SCN2A, SCN8A, SCN9A, SIAT9, SIK1, SLC13A5, SLC25A22, SLC2A1, SLC35A2, SLC6A1, SNIP1, SPTAN1, SRPX2, ST3GAL3, STRADA, STX1B, STXBP1, SYN1, SYNGAP1, SZT2, TBC1D24, and WWOX.

In some embodiments, the methods described herein further comprise identifying a subject having a mutation in one or more of ALDH7A1, ALG13, ARHGEF9, ARX, ASAH1, CDKL5, CHD2, CHRNA2, CHRNA4, CHRNB2, CLN8, CNTNAP2, CPA6, CSTB, DEPDC5, DNM1, EEF1A2, EPM2A, EPM2B, GABRA1, GABRB3, GABRG2, GNAO1, GOSR2, GRIN1, GRIN2A, GRIN2B, HCN1, IER3IP1, KCNA2, KCNB1, KCNC1, KCNMA1, KCNQ2, KCNQ3, KCNT1, KCTD7, LGI1, MEF2C, NHLRC1, PCDH19, PLCB1, PNKP, PNPO, PRICKLE1, PRICKLE2, PRRT2, RELN, SCARB2, SCN1A, SCN1B, SCN2A, SCN8A, SCN9A, SIAT9, SIK1, SLC13A5, SLC25A22, SLC2A1, SLC35A2, SLC6A1, SNIP1, SPTAN1, SRPX2, ST3GAL3, STRADA, STX1B, STXBP1, SYN1, SYNGAP1, SZT2, TBC1D24, and WWOX prior to administration of a compound described herein (e.g., a compound of Formula (I), (I-a), or (I-b), e.g., eleclazine).

Neurodevelopmental Disorders The compounds described herein may be useful in the treatment of a neurodevelopmental disorder. In some embodiments, the neurodevelopmental disorder comprises autism, autism with epilepsy, tuberous sclerosis, Fragile X syndrome, Rett syndrome, Angelman syndrome, Dup15q syndrome, 22q13.3 Deletion syndrome, Prader-Willi syndrome, velocardiofacial syndrome, Smith-Lemli-Opitz syndrome, or a neurodevelopmental disorder with epilepsy. In some embodiments, the methods described herein further comprise identifying a subject having a neurodevelopmental disorder (e.g., autism, autism with epilepsy, tuberous sclerosis, Fragile X syndrome, Rett syndrome, Angelman syndrome, Dup15q syndrome, 22q13.3 Deletion syndrome, Prader-Willi syndrome, velocardiofacial syndrome, Smith-Lemli-Opitz syndrome, or a neurodevelopmental disorder with epilepsy) prior to administration of a compound described herein (e.g., a compound of Formulas (I), (I-a), or (I-b), e.g., eleclazine).

In one aspect, the present invention features a method of treating a neurodevelopmental disorder (e.g., autism, autism with epilepsy, tuberous sclerosis, Fragile X syndrome, Rett syndrome, Angelman syndrome, Dup15q syndrome, 22q13.3 Deletion syndrome, Prader-Willi syndrome, velocardiofacial syndrome, Smith-Lemli-Opitz syndrome, or a neurodevelopmental disorder with epilepsy) comprising administering to a subject in need thereof a compound of Formula (I):

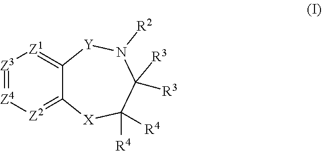

or a pharmaceutically acceptable salt thereof, wherein X, Y, Z¹, Z², Z³, Z⁴, R², R³, R⁴ and subvariables thereof are as defined herein.

In some embodiments, Z¹ is CR⁷ (e.g., CH). In some embodiments, Z² is CR⁷ (e.g., CH). In some embodiments, each of Z¹ and Z² is independently CR⁷ (e.g., CH).

In some embodiments, Z³ is C-Q-R¹. In some embodiments, Q is a covalent bond. In some embodiments, R¹ is aryl (e.g., phenyl) optionally substituted with —O—R²⁰ (e.g., —OCF₃). In some embodiments, Z⁴ is CR⁷ (e.g., CH).

In some embodiments, Y is —C(O)—. In some embodiments, X is —O—.

In some embodiments, R² is —C₁₋₆ alkylene-R⁵. In some embodiments, R² is —CH₂—R⁵.

In some embodiments, R⁵ is heteroaryl (e.g., a nitrogen-containing heteroaryl, e.g., pyrimidinyl).

In some embodiments, each R³ is independently hydrogen. In some embodiments, each R⁴ is independently hydrogen. In some embodiments, each R³ and R⁴ is independently hydrogen.

In some embodiments, the compound of Formula (I) is 4-(pyrimidin-2-ylmethyl)-7-(4-(trifluoromethoxy)phenyl)-3,4-dihydrobenzo[f][1,4]oxazepin-5(2H)-one (i.e., eleclazine, GS-6615, CAS 1443211-72-0), or a pharmaceutically acceptable salt, ester, stereoisomer, mixture of stereoisomers or tautomer thereof. In some embodiments, the compound of Formula (I) is

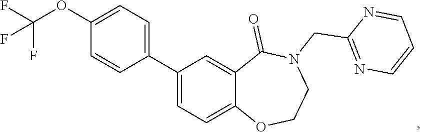 [SMILES: O=C1c2cc(-c3ccc(OC(F)(F)F)cc3)ccc2OCCN1Cc1ncccn1]

or a pharmaceutically acceptable salt, ester, stereoisomer, mixture of stereoisomers or tautomer thereof.

### Pain

The compounds described herein may be useful in the treatment of pain. In some embodiments, the pain comprises neuropathic pain, trigeminal neuralgia, migraine, hemiplegic migraine, familial hemiplegic migraine, familial hemiplegic migraine type 3, cluster headache, trigeminal neuralgia, cerebellar ataxia, or a related headache disorder. In some embodiments, the methods described herein further comprise identifying a subject having pain (e.g., neuropathic pain, trigeminal neuralgia, migraine, hemiplegic migraine, familial hemiplegic migraine, familial hemiplegic migraine type 3, cluster headache, trigeminal neuralgia, cerebellar ataxia, or a related headache disorder) prior to administration of a compound described herein (e.g., a compound of Formulas (I), (I-a), or (I-b), e.g., eleclazine).

In In one aspect, the present invention features a method of treating pain (e.g., neuropathic pain, trigeminal neuralgia, migraine, hemiplegic migraine, familial hemiplegic migraine, familial hemiplegic migraine type 3, cluster headache, trigeminal neuralgia, cerebellar ataxia, or a related headache disorder) comprising administering to a subject in need thereof a compound of Formula (I):

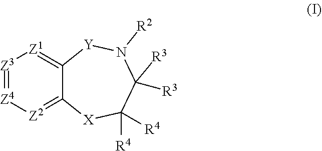

or a pharmaceutically acceptable salt thereof, wherein X, Y, Z¹, Z², Z³, Z⁴, R², R³, R⁴ and subvariables thereof are as defined herein.

In some embodiments, Z¹ is CR⁷ (e.g., CH). In some embodiments, Z² is CR⁷ (e.g., CH). In some embodiments, each of Z¹ and Z² is independently CR⁷ (e.g., CH).

In some embodiments, Z³ is C-Q-R¹. In some embodiments, Q is a covalent bond. In some embodiments, R¹ is aryl (e.g., phenyl) optionally substituted with —O—R²⁰ (e.g., —OCF₃).

In some embodiments, Z⁴ is CR⁷ (e.g., CH).

In some embodiments, Y is —C(O)—. In some embodiments, X is —O—.

In some embodiments, R² is —C₁₋₆ alkylene-R⁵. In some embodiments, R² is —CH₂—R⁵. In some embodiments, R⁵ is heteroaryl (e.g., a nitrogen-containing heteroaryl, e.g., pyrimidinyl).

In some embodiments, each R³ is independently hydrogen. In some embodiments, each R⁴ is independently hydrogen. In some embodiments, each R³ and R⁴ is independently hydrogen.

In some embodiments, the compound of Formula (I) is 4-(pyrimidin-2-ylmethyl)-7-(4-(trifluoromethoxy)phenyl)-3,4-dihydrobenzo[f][1,4]oxazepin-5(2H)-one (i.e., eleclazine, GS-6615, CAS 1443211-72-0), or a pharmaceutically acceptable salt, ester, stereoisomer, mixture of stereoisomers or tautomer thereof. In some embodiments, the compound of Formula (I) is

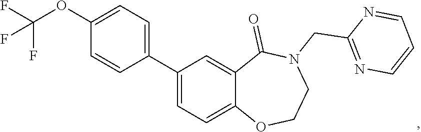 [SMILES: O=C1c2cc(-c3ccc(OC(F)(F)F)cc3)ccc2OCCN1Cc1ncccn1]

or a pharmaceutically acceptable salt, ester, stereoisomer, mixture of stereoisomers or tautomer thereof.

### Neuromuscular Disorders

The compounds described herein may be useful in the treatment of a neuromuscular disorder. In some embodiments, the neuromuscular disorder comprises amyotrophic lateral sclerosis, multiple sclerosism, myotonia, paramyotonia congenita, potassium-aggravated myotonia, periodic paralysis, hyperkalemic periodic paralysis, hypokalemic periodic paralysis, or laryngospasm with SCN4A mutation. In some embodiments, the methods described herein further comprise identifying a subject having a neuromuscular disorder (e.g., amyotrophic lateral sclerosis, multiple sclerosism, myotonia, paramyotonia congenita, potassium-aggravated myotonia, periodic paralysis, hyperkalemic periodic paralysis, hypokalemic periodic paralysis, or laryngospasm with SCN4A mutation) prior to administration of a compound described herein (e.g., a compound of Formulas (I), (I-a), or (I-b), e.g., eleclazine).

In one aspect, the present invention features a method of treating a neuromuscular disorder (e.g., amyotrophic lateral sclerosis, multiple sclerosism, myotonia, paramyotonia congenita, potassium-aggravated myotonia, periodic paralysis, hyperkalemic periodic paralysis, hypokalemic periodic paralysis, or laryngospasm with SCN4A mutation) comprising administering to a subject in need thereof a compound of Formula (I):

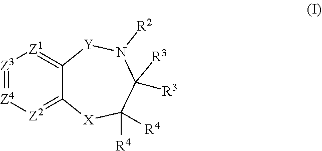

or a pharmaceutically acceptable salt thereof, wherein X, Y, Z¹, Z², Z³, Z⁴, R², R³, R⁴ and subvariables thereof are as defined herein.

In some embodiments, Z¹ is CR⁷ (e.g., CH). In some embodiments, Z² is CR⁷ (e.g., CH). In some embodiments, each of Z¹ and Z² is independently CR⁷ (e.g., CH).

In some embodiments, Z³ is C-Q-R¹. In some embodiments, Q is a covalent bond. In some embodiments, R¹ is aryl (e.g., phenyl) optionally substituted with —O—R²⁰ (e.g., —OCF₃).

In some embodiments, Z⁴ is CR⁷ (e.g., CH).

In some embodiments, Y is —C(O)—. In some embodiments, X is —O—.

In some embodiments, R² is —C₁₋₆ alkylene-R⁵. In some embodiments, R² is —CH₂—R⁵. In some embodiments, R⁵ is heteroaryl (e.g., a nitrogen-containing heteroaryl, e.g., pyrimidinyl).

In some embodiments, each R³ is independently hydrogen. In some embodiments, each R⁴ is independently hydrogen. In some embodiments, each R³ and R⁴ is independently hydrogen.

In some embodiments, the compound of Formula (I) is 4-(pyrimidin-2-ylmethyl)-7-(4-(trifluoromethoxy)phenyl)-3,4-dihydrobenzo[f][1,4]oxazepin-5(2H)-one (i.e., eleclazine, GS-6615, CAS 1443211-72-0), or a pharmaceutically acceptable salt, ester, stereoisomer, mixture of stereoisomers or tautomer thereof. In some embodiments, the compound of Formula (I) is

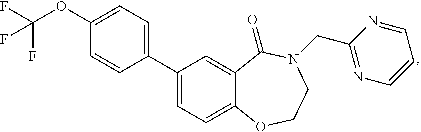 [SMILES: O=C1c2cc(-c3ccc(OC(F)(F)F)cc3)ccc2OCCN1Cc1ncccn1]

or a pharmaceutically acceptable salt, ester, stereoisomer, mixture of stereoisomers or tautomer thereof.

### Other Disorders

In some embodiments, a compound of the present invention (e.g., a compound of Formulas (I), (I-a), or (I-b), e.g., eleclazine) may have appropriate pharmacokinetic properties such that they may active with regard to the central and/or peripheral nervous system. In some embodiments, the compounds provided herein are used to treat a cardiovascular disease such as atrial and ventricular arrhythmias, including atrial fibrillation, Prinzmetal's (variant) angina, stable angina, unstable angina, ischemia and reperfusion injury in cardiac, kidney, liver and the brain, exercise induced angina, pulmonary hypertension, congestive heart disease including diastolic and systolic heart failure, and myocardial infarction. In some embodiments, the compounds provided herein may be used in the treatment of diseases affecting the neuromuscular system resulting in itching, seizures, or paralysis, or in the treatment of diabetes or reduced insulin sensitivity, and disease states related to diabetes, such as diabetic peripheral neuropathy.

In one aspect, the present invention provides a method of treating a neurodevelopmental disorder, wherein the method comprises administering to a subject in need thereof a compound of Formula (I-a):

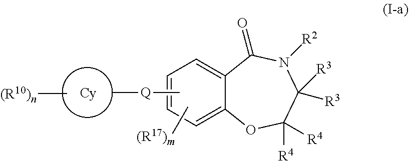

or a pharmaceutically acceptable salt, ester, stereoisomer, mixture of stereoisomers or tautomer thereof, wherein:

Cy is aryl, carbocyclyl, cycloalkenyl, heterocyclyl or heteroaryl;

Q is a covalent bond, —O—C₀₋₂ alkylene, —NR¹¹—C₀₋₂ alkylene, C₂ alkylene, C₂ alkenylene or C₂ alkynylene;

m is 0, 1, 2 or 3;

n is 0, 1, 2, 3, 4 or 5;

each R¹⁰ is independently selected from the group consisting of halo, —NO₂, —CN, —SF₅, —Si(CH₃)₃, —O—R²⁰, —S—R²⁰, —C(O)—R²⁰, —C(O)—OR²⁰, —N(R²⁰)(R²²), —C(O)—N(R²⁰)(R²²), —N(R²⁰)—C(O)—R²², —N(R²⁰)—C(O)—OR²², —N(R²⁰)—S(O)₂—R²⁶, —S(O)₂—R²⁰, —O—S(O)₂—R²⁰, —S(O)₂—N(R²⁰)(R²²), C₁₋₆ alkyl, C₂₋₄ alkenyl, C₂₋₄ alkynyl, carbocyclyl, aryl, heteroaryl and heterocyclyl; and wherein said C₁₋₆ alkyl, C₂₋₄ alkenyl, C₂₋₄ alkynyl, carbocyclyl, aryl, heteroaryl or heterocyclyl are optionally substituted with one, two or three substituents independently selected from the group consisting of halo, —NO₂, aryl, heterocyclyl, heteroaryl, C₁₋₆ alkyl, C₁₋₃ haloalkyl, carbocyclyl, —N(R²⁰)(R²²), —C(O)—R²⁰, —C(O)—OR²⁰, —C(O)—N(R²⁰)(R²²), —CN and —O—R²⁰;

R² is —C₁₋₆ alkylene-R⁵, -L-R⁵, -L-C₁₋₆ alkylene-R⁵, —C₁₋₆ alkylene-L-R⁵ or —C₁₋₆ alkylene-L-C₁₋₆ alkylene-R⁵; wherein each —C₁₋₆ alkyl ene is optionally substituted by one substituent independently selected from the group consisting of C₂₋₄ alkynyl, halo, —NO₂, —CN, —O—R²⁰, —N(R²⁰)(R²²), —C(O)—R²⁰, —C(O)—OR²⁶, —C(O)—N(R²⁰)(R²²), —N(R²⁰)—S(O)₂—R²⁰, carbocyclyl, aryl, heteroaryl or heterocyclyl; and wherein said carbocyclyl, aryl, heteroaryl or heterocyclyl are optionally substituted with one, two or three substituents independently selected from the group consisting of C₁₋₆ alkyl, C₂₋₄ alkynyl, halo, —NO₂, carbocyclyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰;

L is —O—, —S—, —C(O)—, —NHS(O)₂—, —S(O)₂NH—, —C(O)NH— or —NHC(O)—; provided that when R is -L-R or -L-C₁₋₆ alkylene-R, then L is not —O—, —S—, —NHS(O)₂— or —NHC(O)—; and

each R³ is independently hydrogen, deuterium, C₁₋₆ alkyl, carbocyclyl, aryl, heteroaryl or heterocyclyl; wherein said C₁₋₆ alkyl is optionally substituted with one, two or three substituents independently selected from the group consisting of halo, —NO₂, carbocyclyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰; wherein said carbocyclyl, aryl, heterocyclyl or heteroaryl are optionally further substituted with one, two or three substituents independently selected from the group consisting of halo, —NO₂, C₁₋₆ alkyl, aralkyl, carbocyclyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰; and wherein said C₁₋₆ alkyl, aralkyl, carbocyclyl, aryl, heterocyclyl or heteroaryl, are optionally further substituted with one, two or three substituents independently selected from the group consisting of halo, —NO₂, —N(R²⁰)(R²²), —C(O)—R²⁰, —C(O)—OR²⁰, —C(O)—N(R²⁰)(R²²), —CN and —O—R²⁰;

or R² and one of R³ can join together with the atoms to which they are attached to form a heterocyclyl; wherein said heterocyclyl is optionally substituted with one, two or three substituents independently selected from the group consisting of C₁₋₆ alkyl, —O—R²⁰, —N(R²⁰)(R²²), —N(R²⁰)—C(O)—OR²⁰ and —C(O)—OR²⁰; and wherein said C₁₋₆ alkyl is optionally substituted with one, two or three substituents independently selected from the group consisting of halo and heteroaryl;

each R⁴ is independently hydrogen, deuterium, C₁₋₆ alkyl, —C(O)—OR²⁶; —C(O)—N(R²⁶)(R²⁶), carbocyclyl, aryl, heteroaryl or heterocyclyl; wherein said C₁₋₆ alkyl is optionally substituted with one, two or three substituents independently selected from the group consisting of halo, —NO₂, carbocyclyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰; wherein said carbocyclyl, aryl, heterocyclyl or heteroaryl are optionally further substituted with one, two or three substituents independently selected from the group consisting of halo, —NO₂, C₁₋₆ alkyl, aralkyl, carbocyclyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰; and wherein said C₁₋₆ alkyl, aralkyl, carbocyclyl, aryl, heterocyclyl, heteroaryl, are optionally further substituted with one, two or three substituents independently selected from the group consisting of hydroxyl, halo, —NO₂, —N(R²⁰)(R²²), —C(O)—R²⁰, —C(O)—OR²⁰, —C(O)—N(R²⁰)(R²²), —CN and —O—R²⁰;

R⁵ is carbocyclyl, aryl, heteroaryl or heterocyclyl; wherein said carbocyclyl, aryl, heteroaryl or heterocyclyl are optionally substituted with one, two or three substituents independently selected from the group consisting of C₁₋₆ alkyl, C₂₋₄ alkynyl, halo, —NO₂, carbocyclyl, aryl, heterocyclyl, heteroaryl, —N(R²⁰)(R²²), —N(R²⁰)—S(O)₂—R²⁰, —N(R²⁰)—C(O)—R²², —C(O)—R²⁰, —C(O)—OR²⁰, —C(O)—N(R²⁰)(R²²), —CN, oxo and —O— wherein said C₁₋₆ alkyl, carbocyclyl, aryl, heterocyclyl or heteroaryl are optionally further substituted with one, two or three substituents independently selected from the group consisting of halo, —NO₂, C₁₋₆ alkyl, carbocyclyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰; and wherein said C₁₋₆ alkyl, carbocyclyl, aryl, heterocyclyl or heteroaryl are optionally further substituted with one, two or three substituents independently selected from the group consisting of halo, aryl, —NO₂, —CF₃, —N(R²⁰)(R²²), —C(O)—R²⁰, —C(O)—OR²⁰, —C(O)—N(R²⁰)(R²²), —CN, —S(O)₂—R²⁰ and —O—R²⁰;

R¹⁷ is halo, —O—R²⁰ or C₁₋₆ alkyl;

R²⁰ and R²² are in each instance independently selected from the group consisting of hydrogen, C₁₋₆ alkyl, C₂₋₆ alkenyl, C₂₋₆ alkynyl, carbocyclyl, heterocyclyl, aryl and heteroaryl; wherein the C₁₋₆ alkyl, C₂₋₆ alkenyl, C₂₋₆ alkynyl, carbocyclyl, heterocyclyl, aryl and heteroaryl are optionally substituted with one, two or three substituents independently selected from the group consisting of hydroxyl, halo, C₁₋₄ alkyl, acylamino, oxo, —NO₂, —S(O)₂R²⁶, —CN, C₁₋₃ alkoxy, —CF₃, —OCF₃, —OCH₂CF₃, —C(O)—NH₂, aryl, carbocyclyl and heteroaryl; and wherein said heteroaryl is optionally further substituted with C₁₋₄ alkyl or carbocyclyl; or when R²⁰ and R²² are attached to a common nitrogen atom, R²⁰ and R²² may join to form a heterocyclic or heteroaryl ring which is then optionally substituted with one, two or three substituents independently selected from the group consisting of hydroxyl, halo, C₁₋₄ alkyl, aralkyl, aryloxy, aralkyloxy, acylamino, —NO₂, —S(O)₂R²⁶, —CN, C₁₋₄ alkoxy, —CF₃, —OCF₃, aryl, heteroaryl and carbocyclyl; and each R²⁶ is independently selected from the group consisting of hydrogen, C₁₋₄ alkyl, aryl and carbocyclyl; wherein the C₁₋₄ alkyl, aryl and carbocyclyl may be further substituted with from 1 to 3 substituents independently selected from the group consisting of hydroxyl, halo, C₁₋₄ alkoxy, —CF₃ and —OCF₃.

In another aspect, the present invention provides a method of treating a pediatric epilepsy, wherein the method comprises administering to a subject in need thereof a compound of Formula (I-a):

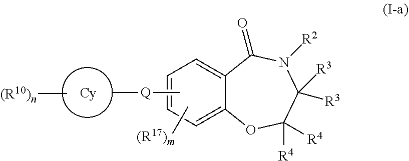

or a pharmaceutically acceptable salt, ester, stereoisomer, mixture of stereoisomers or tautomer thereof, wherein:

Cy is aryl, carbocyclyl, cycloalkenyl, heterocyclyl or heteroaryl;

Q is a covalent bond, —O—C₀₋₂ alkylene, —NR¹¹—C₀₋₂ alkylene, C₂ alkylene, C₂ alkenylene or C₂ alkynylene;

m is 0, 1, 2 or 3;

n is 0, 1, 2, 3, 4 or 5;

each R¹⁰ is independently selected from the group consisting of halo, —NO₂, —CN, —SF₅, —Si(CH₃)₃, —O—R²⁰, —S—R²⁰, —C(O)—R²⁰, —C(O)—OR²⁰, —N(R²⁰)(R²²), —C(O)—N(R²⁰)(R²²), —N(R²⁰)—C(O)—R²², —N(R²⁰)—C(O)—OR²², —N(R²⁰)—S(O)₂—R²⁶, —S(O)₂—R²⁰, —O—S(O)₂—R²⁰, —S(O)₂—N(R²⁰)(R²²), C₁₋₆ alkyl, C₂₋₄ alkenyl, C₂₋₄ alkynyl, carbocyclyl, aryl, heteroaryl and heterocyclyl; and wherein said C₁₋₆ alkyl, C₂₋₄ alkenyl, C₂₋₄ alkynyl, carbocyclyl, aryl, heteroaryl or heterocyclyl are optionally substituted with one, two or three substituents independently selected from the group consisting of halo, —NO₂, aryl, heterocyclyl, heteroaryl, C₁₋₆ alkyl, C₁_₃ haloalkyl, carbocyclyl, —N(R²⁰)(R²²), —C(O)—R²⁰, —C(O)—OR²⁰, —C(O)—N(R²⁰)(R²²), —CN and —O—R²⁰;

R² is —C₁₋₆ alkylene-R⁵, -L-R⁵, -L-C₁₋₆ alkylene-R⁵, —C₁₋₆ alkylene-L-R⁵ or —C₁₋₆ alkylene-L-C₁₋₆ alkylene-R⁵; wherein each —C₁₋₆ alkyl ene is optionally substituted by one substituent independently selected from the group consisting of C₂₋₄ alkynyl, halo, —NO₂, —CN, —O—R²⁰, —N(R²⁰)(R²²), —C(O)—R²⁰, —C(O)—OR²⁶, —C(O)—N(R²⁰)(R²²), —N(R²⁰)—S(O)₂—R²⁰, carbocyclyl, aryl, heteroaryl or heterocyclyl; and wherein said carbocyclyl, aryl, heteroaryl or heterocyclyl are optionally substituted with one, two or three substituents independently selected from the group consisting of C₁₋₆ alkyl, C₂₋₄ alkynyl, halo, —NO₂, carbocyclyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰;

L is —O—, —S—, —C(O)—, —NHS(O)₂—, —S(O)₂NH—, —C(O)NH— or —NHC(O)—; provided that when R is -L-R or -L-C₁₋₆ alkylene-R, then L is not —O—, —S—, —NHS(O)₂— or —NHC(O)—; and

each R³ is independently hydrogen, deuterium, C₁₋₆ alkyl, carbocyclyl, aryl, heteroaryl or heterocyclyl; wherein said C₁₋₆ alkyl is optionally substituted with one, two or three substituents independently selected from the group consisting of halo, —NO₂, carbocyclyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰; wherein said carbocyclyl, aryl, heterocyclyl or heteroaryl are optionally further substituted with one, two or three substituents independently selected from the group consisting of halo, —NO₂, C₁₋₆ alkyl, aralkyl, carbocyclyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰; and wherein said C₁₋₆ alkyl, aralkyl, carbocyclyl, aryl, heterocyclyl or heteroaryl, are optionally further substituted with one, two or three substituents independently selected from the group consisting of halo, —NO₂, —N(R²⁰)(R²²), —C(O)—R²⁰, —C(O)—OR²⁰, —C(O)—N(R²⁰)(R²²), —CN and —O—R²⁰;

or R² and one of R³ can join together with the atoms to which they are attached to form a heterocyclyl; wherein said heterocyclyl is optionally substituted with one, two or three substituents independently selected from the group consisting of C₁₋₆ alkyl, —O—R²⁰, —N(R²⁰)(R²²), —N(R²⁰)—C(O)—OR²⁰ and —C(O)—OR²⁰; and wherein said C₁₋₆ alkyl is optionally substituted with one, two or three substituents independently selected from the group consisting of halo and heteroaryl;

each R⁴ is independently hydrogen, deuterium, C₁₋₆ alkyl, —C(O)—OR²⁶; —C(O)—N(R²⁶)(R²⁶), carbocyclyl, aryl, heteroaryl or heterocyclyl; wherein said C₁₋₆ alkyl is optionally substituted with one, two or three substituents independently selected from the group consisting of halo, —NO₂, carbocyclyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰; wherein said carbocyclyl, aryl, heterocyclyl or heteroaryl are optionally further substituted with one, two or three substituents independently selected from the group consisting of halo, —NO₂, C₁₋₆ alkyl, aralkyl, carbocyclyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰; and wherein said C₁₋₆ alkyl, aralkyl, carbocyclyl, aryl, heterocyclyl, heteroaryl, are optionally further substituted with one, two or three substituents independently selected from the group consisting of hydroxyl, halo, —NO₂, —N(R²⁰)(R²²), —C(O)—R²⁰, —C(O)—OR²⁰, —C(O)—N(R²⁰)(R²²), —CN and —O—R²⁰;

R⁵ is carbocyclyl, aryl, heteroaryl or heterocyclyl; wherein said carbocyclyl, aryl, heteroaryl or heterocyclyl are optionally substituted with one, two or three substituents independently selected from the group consisting of C₁₋₆ alkyl, C₂₋₄ alkynyl, halo, —NO₂, carbocyclyl, aryl, heterocyclyl, heteroaryl, —N(R²⁰)(R²²), —N(R²⁰)—S(O)₂—R²⁰, —N(R²⁰)—C(O)—R²², —C(O)—R²⁰, —C(O)—OR²⁰, —C(O)—N(R²⁰)(R²²), —CN, oxo and —O— wherein said C₁₋₆ alkyl, carbocyclyl, aryl, heterocyclyl or heteroaryl are optionally further substituted with one, two or three substituents independently selected from the group consisting of halo, —NO₂, C₁₋₆ alkyl, carbocyclyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰; and wherein said C₁₋₆ alkyl, carbocyclyl, aryl, heterocyclyl or heteroaryl are optionally further substituted with one, two or three substituents independently selected from the group consisting of halo, aryl, —NO₂, —CF₃, —N(R²⁰)(R²²), —C(O)—R²⁰, —C(O)—OR²⁰, —C(O)—N(R²⁰)(R²²), —CN, —S(O)₂—R²⁰ and —O—R²⁰;

R¹⁷ is halo, —O—R²⁰ or C₁₋₆ alkyl;

R²⁰ and R²² are in each instance independently selected from the group consisting of hydrogen, C₁₋₆ alkyl, C₂₋₆ alkenyl, C₂₋₆ alkynyl, carbocyclyl, heterocyclyl, aryl and heteroaryl; wherein the C₁₋₆ alkyl, C₂₋₆ alkenyl, C₂₋₆ alkynyl, carbocyclyl, heterocyclyl, aryl and heteroaryl are optionally substituted with one, two or three substituents independently selected from the group consisting of hydroxyl, halo, C₁₋₄ alkyl, acylamino, oxo, —NO₂, —S(O)₂R²⁶, —CN, C₁₋₃ alkoxy, —CF₃, —OCF₃, —OCH₂CF₃, —C(O)—NH₂, aryl, carbocyclyl and heteroaryl; and wherein said heteroaryl is optionally further substituted with C₁₋₄ alkyl or carbocyclyl; or when R²⁰ and R²² are attached to a common nitrogen atom, R²⁰ and R²² may join to form a heterocyclic or heteroaryl ring which is then optionally substituted with one, two or three substituents independently selected from the group consisting of hydroxyl, halo, C₁₋₄ alkyl, aralkyl, aryloxy, aralkyloxy, acylamino, —NO₂, —S(O)₂R²⁶, —CN, C₁₋₄ alkoxy, —CF₃, —OCF₃, aryl, heteroaryl and carbocyclyl; and each R²⁶ is independently selected from the group consisting of hydrogen, C₁₋₄ alkyl, aryl and carbocyclyl; wherein the C₁₋₄ alkyl, aryl and carbocyclyl may be further substituted with from 1 to 3 substituents independently selected from the group consisting of hydroxyl, halo, C₁₋₄ alkoxy, —CF₃ and —OCF₃.

In another aspect, the present invention provides a method of treating a refractory epilepsy, wherein the method comprises administering to a subject in need thereof a compound of Formula (I-a):

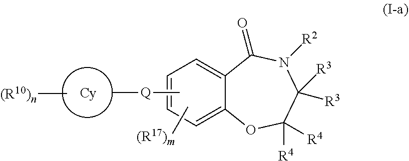

or a pharmaceutically acceptable salt, ester, stereoisomer, mixture of stereoisomers or tautomer thereof, wherein:

Cy is aryl, carbocyclyl, cycloalkenyl, heterocyclyl or heteroaryl;

Q is a covalent bond, —O—C₀₋₂ alkylene, —NR¹¹— C₀₋₂ alkylene, C₂ alkylene, C₂ alkenylene or C₂ alkynylene;

m is 0, 1, 2 or 3;

n is 0, 1, 2, 3, 4 or 5;

each R¹⁰ is independently selected from the group consisting of halo, —NO₂, —CN, —SF₅, —Si(CH₃)₃, —O—R²⁰, —S—R²⁰, —C(O)—R²⁰, —C(O)—OR²⁰, —N(R²⁰)(R²²), —C(O)—N(R²⁰)(R²²), —N(R²⁰)—C(O)—R²², —N(R²⁰)—C(O)—OR²², —N(R²⁰)—S(O)₂—R²⁶, —S(O)₂—R²⁰, —O—S(O)₂—R²⁰, —S(O)₂—N(R²⁰)(R²²), C₁₋₆ alkyl, C₂₋₄ alkenyl, C₂₋₄ alkynyl, carbocyclyl, aryl, heteroaryl and heterocyclyl; and wherein said C₁₋₆ alkyl, C₂₋₄ alkenyl, C₂₋₄ alkynyl, carbocyclyl, aryl, heteroaryl or heterocyclyl are optionally substituted with one, two or three substituents independently selected from the group consisting of halo, —NO₂, aryl, heterocyclyl, heteroaryl, C₁₋₆ alkyl, C₁₋₃ haloalkyl, carbocyclyl, —N(R²⁰)(R²²), —C(O)—R²⁰, —C(O)—OR²⁰, —C(O)—N(R²⁰)(R²²), —CN and —O—R²⁰;

R² is —C₁₋₆ alkylene-R⁵, -L-R⁵, -L-C₁₋₆ alkylene-R⁵, —C₁₋₆ alkylene-L-R⁵ or —C₁₋₆ alkylene-L-C₁₋₆ alkylene-R⁵; wherein each —C₁₋₆ alkyl ene is optionally substituted by one substituent independently selected from the group consisting of C₂₋₄ alkynyl, halo, —NO₂, —CN, —O—R²⁰, —N(R²⁰)(R²²), —C(O)—R²⁰, —C(O)—OR²⁶, —C(O)—N(R²⁰)(R²²), —N(R²⁰)—S(O)₂—R²⁰, carbocyclyl, aryl, heteroaryl or heterocyclyl; and wherein said carbocyclyl, aryl, heteroaryl or heterocyclyl are optionally substituted with one, two or three substituents independently selected from the group consisting of C₁₋₆ alkyl, C₂₋₄ alkynyl, halo, —NO₂, carbocyclyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰;

L is —O—, —S—, —C(O)—, —NHS(O)₂—, —S(O)₂NH—, —C(O)NH— or —NHC(O)—; provided that when R is -L-R or -L-C₁₋₆ alkylene-R, then L is not —O—, —S—, —NHS(O)₂— or —NHC(O)—; and

each R³ is independently hydrogen, deuterium, C₁₋₆ alkyl, carbocyclyl, aryl, heteroaryl or heterocyclyl; wherein said C₁₋₆ alkyl is optionally substituted with one, two or three substituents independently selected from the group consisting of halo, —NO₂, carbocyclyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰; wherein said carbocyclyl, aryl, heterocyclyl or heteroaryl are optionally further substituted with one, two or three substituents independently selected from the group consisting of halo, —NO₂, C₁₋₆ alkyl, aralkyl, carbocyclyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰; and wherein said C₁₋₆ alkyl, aralkyl, carbocyclyl, aryl, heterocyclyl or heteroaryl, are optionally further substituted with one, two or three substituents independently selected from the group consisting of halo, —NO₂, —N(R²⁰)(R²²), —C(O)—R²⁰, —C(O)—OR²⁰, —C(O)—N(R²⁰)(R²²), —CN and —O—R²⁰;

or R² and one of R³ can join together with the atoms to which they are attached to form a heterocyclyl; wherein said heterocyclyl is optionally substituted with one, two or three substituents independently selected from the group consisting of C₁₋₆ alkyl, —O—R²⁰, —N(R²⁰)(R²²), —N(R²⁰)—C(O)—OR²⁰ and —C(O)—OR²⁰; and wherein said C₁₋₆ alkyl is optionally substituted with one, two or three substituents independently selected from the group consisting of halo and heteroaryl;

each R⁴ is independently hydrogen, deuterium, C₁₋₆ alkyl, —C(O)—OR²⁶; —C(O)—N(R²⁶)(R²⁶), carbocyclyl, aryl, heteroaryl or heterocyclyl; wherein said C₁₋₆ alkyl is optionally substituted with one, two or three substituents independently selected from the group consisting of halo, —NO₂, carbocyclyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰; wherein said carbocyclyl, aryl, heterocyclyl or heteroaryl are optionally further substituted with one, two or three substituents independently selected from the group consisting of halo, —NO₂, C₁₋₆ alkyl, aralkyl, carbocyclyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰; and wherein said C₁₋₆ alkyl, aralkyl, carbocyclyl, aryl, heterocyclyl, heteroaryl, are optionally further substituted with one, two or three substituents independently selected from the group consisting of hydroxyl, halo, —NO₂, —N(R²⁰)(R²²), —C(O)—R²⁰, —C(O)—OR²⁰, —C(O)—N(R²⁰)(R²²), —CN and —O—R²⁰;

R⁵ is carbocyclyl, aryl, heteroaryl or heterocyclyl; wherein said carbocyclyl, aryl, heteroaryl or heterocyclyl are optionally substituted with one, two or three substituents independently selected from the group consisting of C₁₋₆ alkyl, C₂₋₄ alkynyl, halo, —NO₂, carbocyclyl, aryl, heterocyclyl, heteroaryl, —N(R²⁰)(R²²), —N(R²⁰)—S(O)₂—R²⁰, —N(R²⁰)—C(O)—R²², —C(O)—R²⁰, —C(O)—OR²⁰, —C(O)—N(R²⁰)(R²²), —CN, oxo and —O— wherein said C₁₋₆ alkyl, carbocyclyl, aryl, heterocyclyl or heteroaryl are optionally further substituted with one, two or three substituents independently selected from the group consisting of halo, —NO₂, C₁₋₆ alkyl, carbocyclyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰; and wherein said C₁₋₆ alkyl, carbocyclyl, aryl, heterocyclyl or heteroaryl are optionally further substituted with one, two or three substituents independently selected from the group consisting of halo, aryl, —NO₂, —CF₃, —N(R²⁰)(R²²), —C(O)—R²⁰, —C(O)—OR²⁰, —C(O)—N(R²⁰)(R²²), —CN, —S(O)₂—R²⁰ and —O—R²⁰;

R¹⁷ is halo, —O—R²⁰ or C₁₋₆ alkyl;

R²⁰ and R²² are in each instance independently selected from the group consisting of hydrogen, C₁₋₆ alkyl, C₂₋₆ alkenyl, C₂₋₆ alkynyl, carbocyclyl, heterocyclyl, aryl and heteroaryl; wherein the C₁₋₆ alkyl, C₂₋₆ alkenyl, C₂₋₆ alkynyl, carbocyclyl, heterocyclyl, aryl and heteroaryl are optionally substituted with one, two or three substituents independently selected from the group consisting of hydroxyl, halo, C₁₋₄ alkyl, acylamino, oxo, —NO₂, —S(O)₂R²⁶, —CN, C₁₋₃ alkoxy, —CF₃, —OCF₃, —OCH₂CF₃, —C(O)—NH₂, aryl, carbocyclyl and heteroaryl; and wherein said heteroaryl is optionally further substituted with C₁₋₄ alkyl or carbocyclyl; or when R²⁰ and R²² are attached to a common nitrogen atom, R²⁰ and R²² may join to form a heterocyclic or heteroaryl ring which is then optionally substituted with one, two or three substituents independently selected from the group consisting of hydroxyl, halo, C₁₋₄ alkyl, aralkyl, aryloxy, aralkyloxy, acylamino, —NO₂, —S(O)₂R²⁶, —CN, C₁₋₄ alkoxy, —CF₃, —OCF₃, aryl, heteroaryl and carbocyclyl; and each R²⁶ is independently selected from the group consisting of hydrogen, C₁₋₄ alkyl, aryl and carbocyclyl; wherein the C₁₋₄ alkyl, aryl and carbocyclyl may be further substituted with from 1 to 3 substituents independently selected from the group consisting of hydroxyl, halo, C₁₋₄ alkoxy, —CF₃ and —OCF₃.

In some embodiments, Q is a covalent bond. In some embodiments, Cy is aryl (e.g., phenyl). In some embodiments, R¹⁰ is —O—R²⁰ (e.g., —OCF₃). In some embodiments, n is 1. In some embodiments, m is 0. In some embodiments, R² is —C₁₋₆ alkylene-R⁵ (e.g., —CH₂—R⁵). In some embodiments, R⁵ is heteroaryl (e.g., a nitrogen-containing heteroaryl, e.g., pyrimidinyl). In some embodiments, each R³ is independently hydrogen. In some embodiments, each R⁴ is independently hydrogen.

In some embodiments, the compound of Formula (I-a) is a compound of Formula (I-b):

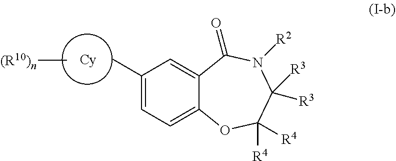

or a pharmaceutically acceptable salt, ester, stereoisomer, mixture of stereoisomers or tautomer thereof, wherein Cy, R², R³, R⁴, R¹⁰, and n are defined as in Formula (I-a).

In some embodiments, the compound of Formula (I-a) or (I-b) is selected from a compound disclosed herein.

In some embodiments, the compound of Formula (I-a) or (I-b) is:

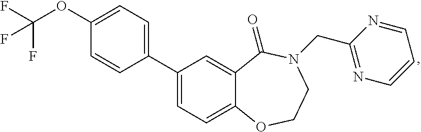 [SMILES: O=C1c2cc(-c3ccc(OC(F)(F)F)cc3)ccc2OCCN1Cc1ncccn1]

or a pharmaceutically acceptable salt, ester, stereoisomer, mixture of stereoisomers or tautomer thereof.

In some embodiments, the neurodevelopmental disorder, pediatric epilepsy, or refractory epilepsy comprises Dravet syndrome or a genetic epilepsy.

In some embodiments, the neurodevelopmental disorder, pediatric epilepsy, or refractory epilepsy is associated with a mutation in ALDH7A1, ALG13, ARHGEF9, ARX, ASAH1, CDKL5, CHD2, CHRNA2, CHRNA4, CHRNB2, CLN8, CNTNAP2, CPA6, CSTB, DEPDC5, DNM1, EEF1A2, EPM2A, EPM2B, GABRA1, GABRB3, GABRG2, GNAO1, GOSR2, GRIN1, GRIN2A, GRIN2B, HCN1, IER3IP1, KCNA2, KCNB1, KCNC1, KCNMA1, KCNQ2, KCNQ3, KCNT1, KCTD7, LGI1, MEF2C, NHLRC1, PCDH19, PLCB1, PNKP, PNPO, PRICKLE1, PRICKLE2, PRRT2, RELN, SCARB2, SCN1A, SCN1B, SCN2A, SCN8A, SCN9A, SIAT9, SIK1, SLC13A5, SLC25A22, SLC2A1, SLC35A2, SLC6A1, SNIP1, SPTAN1, SRPX2, ST3GAL3, STRADA, STX1B, STXBP1, SYN1, SYNGAP1, SZT2, TBC1D24, or WWOX.

In some embodiments, the neurodevelopmental disorder, pediatric epilepsy, or refractory epilepsy is associated with a mutation in SCN2A.

In some embodiments, the neurodevelopmental disorder, pediatric epilepsy, or refractory epilepsy is associated with a mutation in SCN8A.

In some embodiments, the method further comprises administering the compound of formula I-a or I-b to a subject identified as having a mutation in SCN2A.

In some embodiments, the method further comprises administering the compound of formula I-a or I-b to a subject identified as having a mutation in SCN8A.

In some embodiments, the method further comprises assaying a sample from a subject to evaluate for the presence of a mutation in SCN2A.

In some embodiments, the method further comprises assaying a sample from a subject to evaluate for the presence of a mutation in SCN8A.

In some embodiments, the epilepsy is characterized by a mutation in an epilepsy associated gene or autism associated gene, the method comprising administering to the subject a modulator (e.g., an inhibitor) of a sodium ion channel to treat the epilepsy.

In one aspect, the present invention provides a method of diagnosing and treating a subject suffering from a epilepsy, the method comprising:
a. diagnosing whether the subject has an epilepsy that is susceptible to a treatment, the treatment comprising administration of a modulator (e.g., an inhibitor) of sodium channel, based on the presence of a mutation in an epilepsy associated gene or autism associated gene previously determined to be present in a sample from the subject; and
b. administering to the subject the treatment in an amount effective to treat the epilepsy.

In some embodiments, the modulator of the sodium channel is eleclazine.

In some embodiments, the mutation is a mutation in the gene selected from ALDH7A1, ALG13, ARHGEF9, ARX, ASAH1, CDKL5, CHD2, CHRNA2, CHRNA4, CHRNB2, CLN8, CNTNAP2, CPA6, CSTB, DEPDC5, DNM1, EEF1A2, EPM2A, EPM2B, GABRA1, GABRB3, GABRG2, GNAO1, GOSR2, GRIN1, GRIN2A, GRIN2B, HCN1, IER3IP1, KCNA2, KCNB1, KCNC1, KCNMA1, KCNQ2, KCNQ3, KCNT1, KCTD7, LGI1, MEF2C, NHLRC1, PCDH19, PLCB1, PNKP, PNPO, PRICKLE1, PRICKLE2, PRRT2, RELN, SCARB2, SCN1A, SCN1B, SCN2A, SCN8A, SCN9A, SIAT9, SIK1, SLC13A5, SLC25A22, SLC2A1, SLC35A2, SLC6A1, SNIP1, SPTAN1, SRPX2, ST3GAL3, STRADA, STX1B, STXBP1, SYN1, SYNGAP1, SZT2, TBC1D24, and WWOX.

In some embodiments, the mutation is a mutation in SCN2A.

In some embodiments, the mutation is a mutation in SCN8A.

In some embodiments, the method further comprises administering a modulator (e.g., an inhibitor) of a sodium ion to a subject identified as having a mutation in SCN2A.

In some embodiments, the method further comprises administering a modulator (e.g., an inhibitor) of a sodium ion to a subject identified as having a mutation in SCN8A.

In some embodiments, the method further comprises assaying a sample from a subject to evaluate for the presence of a mutation in SCN2A.

In some embodiments, the method further comprises assaying a sample from a subject to evaluate for the presence of a mutation in SCN8A.

In some embodiments, the mutation is a spontaneous mutation.

In some embodiments, the mutation is determined in the subject by sequencing of the DNA or RNA from a sample of the subject.

In some embodiments, the sequencing comprises sequencing of the whole genome or whole exome of the subject.

In some embodiments, the sequencing comprises sequencing a panel of genes from the subject.

In some embodiments, the mutation is a mutation in an epilepsy associated gene.

In some embodiments, the mutation is a mutation identified in the Epilepsy phenome/Genome project.

In some embodiments, the mutation is in an autism associated gene.

In some embodiments, the inhibitor is an inhibitor of late sodium current (INaL).

In one aspect, the method for treating a subject having an epilepsy, wherein the subject is characterized as having normal cardiac function, the method comprising administering to the subject a modulator (e.g., an inhibitor) of a sodium ion channel to treat the epilepsy.

In some embodiments, the modulator is a modulator (e.g., an inhibitor) of late sodium current (INaL).

In some embodiments, the subject is characterized as being without structural heart disease.

In some embodiments, the subject is characterized as being without arrhythmias.

In some embodiments, the method further comprises receiving information characterizing the subject based on an electrocardiogram.

In some embodiments, the method further comprises receiving information characterizing the subject based on a cardiac ultrasound.

In some embodiments, the method further comprises receiving information characterizing the subject based on an echocardiogram (e.g., relating to structural heart disease or arrhythmia).

In some embodiments, the method further comprising receiving information characterizing the subject based on a cardiac MRI.

In some embodiments, the method further comprises receiving information characterizing the subject based on a cardiac CT.

### Pharmaceutical Compositions and Routes of Administration

Compounds provided in accordance with the present invention are usually administered in the form of pharmaceutical compositions. This invention therefore provides pharmaceutical compositions that contain, as the active ingredient, one or more of the compounds described, or a pharmaceutically acceptable salt or ester thereof, and one or more pharmaceutically acceptable excipients, carriers, including inert solid diluents and fillers, diluents, including sterile aqueous solution and various organic solvents, permeation enhancers, solubilizers and adjuvants. The pharmaceutical compositions may be administered alone or in combination with other therapeutic agents. Such compositions are prepared in a manner well known in the pharmaceutical art (see, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.)

The pharmaceutical compositions may be administered in either single or multiple doses by any of the accepted modes of administration of agents having similar utilities, for example as described in those patents and patent applications incorporated by reference, including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer.

One mode for administration is parenteral, particularly by injection. The forms in which the novel compositions of the present invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. Aqueous solutions in saline are also conventionally used for injection, but less preferred in the context of the present invention. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating a compound according to the present invention in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Oral administration is another route for administration of compounds in accordance with the invention. Administration may be via capsule or enteric coated tablets, or the like. In making the pharmaceutical compositions that include at least one compound described herein, the active ingredient is usually diluted by an excipient and/or enclosed within such a carrier that can be in the form of a capsule, sachet, paper or other container. When the excipient serves as a diluent, it can be in the form of a solid, semi-solid, or liquid material (as above), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl and propylhydroxy-benzoates; sweetening agents; and flavoring agents.

The compositions of the invention can be formulated so as to provide quick, sustained or delayed release of the active ingredient after administration to the patient by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

The compositions are preferably formulated in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient (e.g., a tablet, capsule, ampoule). The compounds are generally administered in a pharmaceutically effective amount. In some embodiments, for oral administration, each dosage unit contains from about 0.01 mg to about 2 g of a compound described herein (e.g., from about 0.1 mg to about 500 mg), and for parenteral administration, preferably from 0.01 to 700 mg of a compound described herein (e.g., from about 0.1 mg to about 500 mg). It will be understood, however, that the amount of the compound actually administered usually will be determined by a physician, in the light of the relevant circumstances, including the condition to be treated, the chosen route of administration, the actual compound administered and its relative activity, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules.

The tablets or pills of the present invention may be coated or otherwise compounded to provide a dosage form affording the advantage of prolonged action, or to protect from the acid conditions of the stomach. For example, the tablet or pill can comprise an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably, the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, preferably orally or nasally, from devices that deliver the formulation in an appropriate manner.

In some embodiments, the composition is a liquid formulation, e.g., a liquid solution comprising a cyclodextrin, such as a beta cyclodextrin derivative, e.g., a sulfobutyl ether-beta-cyclodextrin, e.g., captisol, as disclosed in International Patent Publication No. WO 2017/007805, which is incorporated herein by reference in its entirety.

In some embodiments, the composition is formulated for administration to a pediatric subject (e.g., a subject under 18 years of age). Pediatric medicines should be formulated to suit the needs of children based on, for example, a child's age, size, physiological condition, and treatment requirements. Children are different from adults in various aspects of pharmacokinetics and pharmacodynamics including potential routes of administration, drug-related toxicity, and taste preferences. For example, the rate of gastric emptying and pH, gastrointestinal permeability, and the surface area available for drug absorption are some of the important differences of pharmacokinetics between children and adults. The age effect on pharmacokinetics also leads to different dosing requirements for different age groups of children. Further, formulation acceptability and preferences are key factors in promoting medication adherence in children. Pain, discomfort, and an unnecessary burden on children and/or caregivers during drug administration should be avoided to ensure medication adherence. Use of tasteless or palatable medicines can help to prevent loss of medication from spillage and/or spitting because children have low tolerance with regard to unpleasant tastes. To treat all children, pediatric formulations should have flexible dosage increments and minimal excipients, be palatable, safe and easy to administer, and be stable with regard to light, humidity, and heat. For example, age-appropriate oral solid preparations which enable dose flexibility, easier administration, and better acceptance in children would be useful as pediatric formulations.

### Patient Diagnosing and Monitoring

The present invention provides a method for diagnosing a epilepsy in a subject that is susceptible to a treatment, wherein the treatment comprises administering a modulator of (e.g., inhibitor of) a sodium ion channel (e.g., late sodium current (INaL), said method comprising:

a. obtaining a biological sample from a human subject;

b. detecting whether a mutation in an epilepsy associated gene or autism associated gene is present in the biological sample; and

c. diagnosing the patient with an epilepsy susceptible to treatment with a comprises a modulator of (e.g., inhibitor of) a sodium ion channel (e.g., late sodium current (INaL) when the presence of a mutation in an epilepsy associated gene or autism associated gene in the biological sample is detected.

The present invention also provides a method for predicting the efficacy of a treatment of a epilepsy in a subject, the treatment comprising administering a modulator of (e.g., inhibitor of) a sodium ion channel (e.g., late sodium current (INaL), the method comprising the step of:

determining if, having determined if, or receiving information that the subject has a mutation in an epilepsy associated gene or autism associated gene, by a method selected from hybridization-based methods, amplification-based methods, microarray analysis, flow cytometry analysis, DNA sequencing, next generation sequencing, primer extension, PCR, in situ hybridization, dot blot, and Southern blot;

wherein said determining if, having determined if, or receiving information is predictive of efficacy of the treatment.

The present invention also provides a method of predicting the efficacy of a treatment of a epilepsy in a subject, the treatment administering a modulator of (e.g., inhibitor of) a sodium ion channel (e.g., late sodium current (INaL), the method comprising the step of:

determining if, having determined if, or receiving information that the subject has a mutation in an epilepsy associated gene or autism associated gene, e.g., by a method selected from hybridization-based methods, amplification-based methods, microarray analysis, flow cytometry analysis, DNA sequencing, next generation sequencing, primer extension, PCR, in situ hybridization, dot blot, and Southern blot;

wherein said determining if, having determined if, or receiving information is predictive that the treatment will not be efficacious in treating the cancer in the subject.

### Patient Sample

The terms “patient sample”, “subject sample”, “biological sample,” and “sample” are used interchangeably herein. The subject sample can be a tissue, or bodily fluid, or bodily product. Tissue samples can include fixed, paraffin embedded, fresh, or frozen samples. For example, the tissue sample can include a biopsy, cheek swab. Exemplary tissues include lung, breast, brain, nervous tissue, kidney, ovary, thyroid, pancreas, colon, prostate, lymph node, skin, hair follicles and nails. Exemplary samples include samples derived from solid tumors.

Exemplary bodily fluids include blood, plasma, urine, lymph, tears, sweat, saliva, semen, and cerebrospinal fluid. Exemplary bodily products include exhaled breath.

The tissue, fluid or product can be removed from the patient and analyzed. The evaluation can include one or more of: performing the analysis of the tissue, fluid or product; requesting analysis of the tissue fluid or product; requesting results from analysis of the tissue, fluid or product; or receiving the results from analysis of the tissue, fluid or product.

### Methods of Evaluating Samples

The presence of a mutation in an epilepsy associated gene or autism associated gene, can be assessed using any of a wide variety of well-known methods for detecting expression of a transcribed molecule, gene, protein, mRNA, genomic DNA, or cDNA. Non-limiting examples of such methods include nucleic acid hybridization-based methods, amplification-based methods, microarray analysis, flow cytometry analysis, DNA sequencing, next generation sequencing, primer extension, PCR, in situ hybridization, dot blot, and Southern blot.

### Kits

Also described herein are kits comprising a means to assay a mutation in an epilepsy associated gene or autism associated gene. For example, the kit can include an agent capable of interacting with a gene expression product of a gene described herein. The kit can include a plurality of agents capable of interacting with gene expression products of a plurality of genes described herein. The agent can include, but is not limited to, an antibody, a plurality of antibodies, an oligonucleotide, or a plurality of oligonucleotides. The gene expression product can include, but is not limited to, a transcribed molecule, a RNA molecule, a polypeptide, a protein, genomic DNA, or cDNA.

### Compounds

Described herein are compounds and compositions thereof and their use to treat subject described herein. In an embodiment, the subject is suffering from a disease or disorder described herein and has been characterized as having normal cardiac function. In an embodiment, the subject is suffering from an epilepsy characterized by a mutation in an epilepsy associated gene or autism associated gene. In some embodiments, a compound provided by the present invention is effective in the treatment of epilepsy or an epilepsy syndrome, a neurodevelopmental disorder, pain, migraine, headache, a neuromuscular disorder, or a psychiatric disorder (e.g., bipolar disorder). Compounds described herein may also modulate all sodium ion channels, or may be specific to only one or a plurality of sodium ion channels, e.g., Nav 1.1, 1.2, 1.4, 1.5, 1.6, 1.7, 1.8, and/or 1.9.

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E. L. Eliel, Ed., Univ. of Notre Dame Press, Notre Dame, Ind. 1972). The invention additionally encompasses compounds described herein as individual isomers substantially free of other isomers, and alternatively, as mixtures of various isomers.

As used herein a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the enantiomer. In certain embodiments, the weights are based upon total weight of all enantiomers or stereoisomers of the compound.

In the compositions provided herein, an enantiomerically pure compound can be present with other active or inactive ingredients. For example, a pharmaceutical composition comprising enantiomerically pure R-compound can comprise, for example, about 90% excipient and about 10% enantiomerically pure R-compound. In certain embodiments, the enantiomerically pure R-compound in such compositions can, for example, comprise, at least about 95% by weight R-compound and at most about 5% by weight S-compound, by total weight of the compound. For example, a pharmaceutical composition comprising enantiomerically pure S-compound can comprise, for example, about 90% excipient and about 10% enantiomerically pure S-compound. In certain embodiments, the enantiomerically pure S-compound in such compositions can, for example, comprise, at least about 95% by weight S-compound and at most about 5% by weight R-compound, by total weight of the compound. In certain embodiments, the active ingredient can be formulated with little or no excipient or carrier.

Compound described herein may also comprise one or more isotopic substitutions. For example, H may be in any isotopic form, including ¹H, ²H (D or deuterium), and ³H (T or tritium); C may be in any isotopic form, including ¹²C, ¹³C, and ¹⁴C; O may be in any isotopic form, including ¹⁶O and ¹⁸O; and the like.

The following terms are intended to have the meanings presented therewith below and are useful in understanding the description and intended scope of the present invention. When describing the invention, which may include compounds, pharmaceutical compositions containing such compounds and methods of using such compounds and compositions, the following terms, if present, have the following meanings unless otherwise indicated. It should also be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein. The articles “a” and “an” may be used herein to refer to one or to more than one (i.e. at least one) of the grammatical objects of the article. By way of example “an analogue” means one analogue or more than one analogue.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

“Alkyl” refers to a radical of a straight-chain or branched saturated hydrocarbon group, e.g., having 1 to 20 carbon atoms (“C₁₋₂₀ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). Examples of C₁₋₆ alkyl groups include methyl, ethyl, propyl, isopropyl, butyl, isobutyl, pentyl, hexyl, and the like.

“Alkenyl” refers to a radical of a straight-chain or branched hydrocarbon group having from 2 to 20 carbon atoms, one or more carbon-carbon double bonds (e.g., 1, 2, 3, or 4 carbon-carbon double bonds), and optionally one or more carbon-carbon triple bonds (e.g., 1, 2, 3, or 4 carbon-carbon triple bonds) (“C₂₋₂₀ alkenyl”). In certain embodiments, alkenyl does not contain any triple bonds. In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like.

“Alkynyl” refers to a radical of a straight-chain or branched hydrocarbon group having from 2 to 20 carbon atoms, one or more carbon-carbon triple bonds (e.g., 1, 2, 3, or 4 carbon-carbon triple bonds), and optionally one or more carbon-carbon double bonds (e.g., 1, 2, 3, or 4 carbon-carbon double bonds) (“C₂₋₂₀ alkynyl”). In certain embodiments, alkynyl does not contain any double bonds. In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like.

As used herein, “alkylene,” “alkenylene,” and “alkynylene,” refer to a divalent radical of an alkyl, alkenyl, and alkynyl group respectively. When a range or number of carbons is provided for a particular “alkylene,” “alkenylene,” or “alkynylene,” group, it is understood that the range or number refers to the range or number of carbons in the linear carbon divalent chain. “Alkylene,” “alkenylene,” and “alkynylene,” groups may be substituted or unsubstituted with one or more substituents as described herein.

“Aryl” refers to a radical of a monocyclic or polycyclic (e.g., bicyclic or tricyclic) 4n+2 aromatic ring system (e.g., having 6, 10, or 14π electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, and trinaphthalene. Particularly aryl groups include phenyl, naphthyl, indenyl, and tetrahydronaphthyl.

“Fused aryl” refers to an aryl having two of its ring carbon in common with a second aryl or heteroaryl ring or with a carbocyclyl or heterocyclyl ring.

“Heteroaryl” refers to a radical of a 5-10 membered monocyclic or bicyclic 4n+2 aromatic ring system (e.g., having 6 or 10π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

Examples of representative heteroaryls include the following:

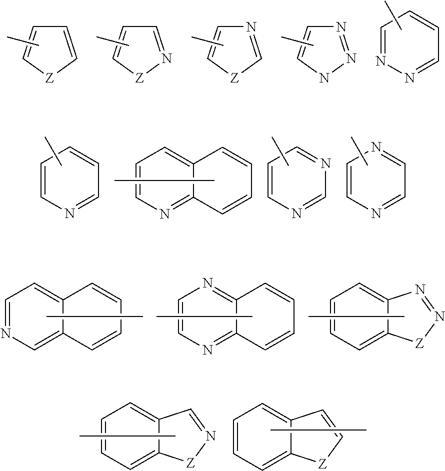

wherein each Z is selected from carbonyl, N, NR⁶⁵, O, and S; and R⁶⁵ is independently hydrogen, C₁-C₈ alkyl, C₃-C₁₀ carbocyclyl, 4-10 membered heterocyclyl, C₆-C₁₀ aryl, and 5-10 membered heteroaryl.

“Aralkyl” refers to an alkyl group substituted with an aryl group. “Aralkyloxy” refers to aryl bound to alkylene bound to oxygen (O). “Aryloxy” refers to aryl bound to oxygen (O).

“Carbocyclyl” or “carbocyclic” refers to a radical of a non-aromatic cyclic hydrocarbon group having from 3 to 10 ring carbon atoms (“C₃₋₁₀ carbocyclyl”) and zero heteroatoms in the non-aromatic ring system. In some embodiments, a carbocyclyl group has 3 to 8 ring carbon atoms (“C₃₋₈ carbocyclyl”). In some embodiments, a carbocyclyl group has 3 to 6 ring carbon atoms (“C₃₋₆ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or contain a fused, bridged or spiro ring system such as a bicyclic system (“bicyclic carbocyclyl”) and can be saturated or can be partially unsaturated. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system.

“Heterocyclyl” or “heterocyclic” refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system.

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like.

Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like. Exemplary 7-membered heterocyclyl groups fused to an aryl ring (also referred to herein as a 7,6-bicyclic heterocyclic ring) include, without limitation, dihydrobenzooxazepinonyl, dihydrobenzooxepinonyl, tetrahydrobenzooxepinyl, tetrahydrobenzoazepinyl, tetrahydrobenzoazepinonyl, tetrahydroisoquinolinyl, and the like.

“Hetero” when used to describe a compound or a group present on a compound means that one or more carbon atoms in the compound or group have been replaced by a nitrogen, oxygen, or sulfur heteroatom. Hetero may be applied to any of the hydrocarbyl groups described above such as alkyl, e.g., heteroalkyl, carbocyclyl, e.g., heterocyclyl, aryl, e.g. heteroaryl, cycloalkenyl, e.g. cycloheteroalkenyl, and the like having from 1 to 5, and particularly from 1 to 3 heteroatoms.

“Cyano” refers to the radical —CN.

“Halo” or “halogen” refers to fluoro (F), chloro (Cl), bromo (Br), and iodo (I). In certain embodiments, the halo group is either fluoro or chloro.

“Haloalkyl” refers to an alkyl group substituted with one or more halogen atoms.

“Nitro” refers to the radical —NO₂.

In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position.

A “counterion” or “anionic counterion” is a negatively charged group associated with a cationic quaternary amino group in order to maintain electronic neutrality. Exemplary counterions include halide ions (e.g., F⁻, Cl⁻, Br⁻, F⁻), NO₃⁻, ClO₄⁻, OH⁻, H₂PO₄⁻, HSO₄⁻, SO₄⁻² sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), and carboxylate ions (e.g., acetate, ethanoate, propanoate, benzoate, glycerate, lactate, tartrate, glycolate, and the like).

Nitrogen atoms can be substituted or unsubstituted as valency permits, and include primary, secondary, tertiary, and quarternary nitrogen atoms. Exemplary nitrogen atom substitutents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups attached to a nitrogen atom are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

These and other exemplary substituents are described in more detail in the Detailed Description, Examples, and Claims. The invention is not intended to be limited in any manner by the above exemplary listing of substituents.

### Other Definitions

The term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

In typical embodiments, the present invention is intended to encompass the compounds disclosed herein, and the pharmaceutically acceptable salts, pharmaceutically acceptable esters, tautomeric forms, polymorphs, and prodrugs of such compounds. In some embodiments, the present invention includes a pharmaceutically acceptable addition salt, a pharmaceutically acceptable ester, a hydrate of an addition salt, a tautomeric form, a polymorph, an enantiomer, a mixture of enantiomers, a stereoisomer or mixture of stereoisomers (pure or as a racemic or non-racemic mixture) of a compound described herein, e.g. a compound of Formula (I), (I-a), or (I-b), e.g., eleclazine.

In one aspect, the present invention features a compound of Formula (I):

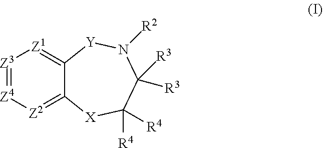

or a pharmaceutically acceptable salt, ester, stereoisomer, mixture of stereoisomers or tautomer thereof, wherein:

Z¹ and Z² are each independently selected from the group consisting of CR⁷ and N;

Z³ and Z⁴ are each independently selected from the group consisting of CR⁷, C-Q-R¹ and N, provided that one of Z³ and Z⁴ is C-Q-R¹ and the other of Z³ and Z⁴ is CR⁷ or N; wherein only one of Z¹, Z², and Z⁴ is N;

X is —O— or —NR⁶—;

Y is —C(O)—, —C(R¹¹)₂— or —S(O)₂—;

Q is a covalent bond, —O—C₀₋₂ alkylene, —NR¹¹—CO₂ alkylene, C₂ alkylene, C₂ alkenylene, or C₂ alkynylene;

R¹ is aryl, carbocyclyl, cycloalkenyl, heterocyclyl or heteroaryl; wherein said aryl, carbocyclyl, cycloalkenyl, heterocyclyl or heteroaryl is optionally substituted with one, two or three substituents independently selected from the group consisting of halo, —NO₂, —CN, —SF₅, —Si(CH₃)₃, —O—R²⁰, —S—R²⁰, —C(O)—R²⁰, —C(O)—OR²⁰, —N(R²⁰)(R²²), —C(O)—N(R²⁰)(R²²), —N(R²⁰)—C(O)—R²², —N(R²⁰)—C(O)—OR²², —N(R²⁰)—S(O)₂—R²⁶, —S(O)₂—R²⁰, —O—S(O)₂—R²⁰, —S(O)₂—N(R²⁰)(R²²), C₁₋₆ alkyl, C₂₋₄ alkenyl, C₂₋₄ alkynyl, carbocyclyl, aryl, heteroaryl and heterocyclyl; and wherein said C₁₋₆ alkyl, C₂₋₄ alkenyl, C₂₋₄ alkynyl, carbocyclyl, aryl, heteroaryl or heterocyclyl are optionally substituted with one, two or three substituents independently selected from the group consisting of halo, —NO₂, aryl, heterocyclyl, heteroaryl, C₁₋₆ alkyl, C₁₋₃ haloalkyl, carbocyclyl, —N(R²⁰)(R²²), —C(O)—R²⁰, —C(O)—OR²⁰, —C(O)—N(R²⁰)(R²²), —CN and —O—R²⁰;

R² is —C₁₋₆ alkylene-R⁵, -L-R⁵, -L-C₁₋₆ alkylene-R⁵, —C₁₋₆ alkylene-L-R⁵ or —C₁₋₆ alkylene-L-C₁₋₆ alkylene-R⁵; wherein each —C₁₋₆ alkylene is optionally substituted by one substituent independently selected from the group consisting of C₂₋₄ alkynyl, halo, —NO₂, —CN, —O—R²⁰, —N(R²⁰)(R²²), —C(O)—R²⁰, —C(O)—OR²⁶, —C(O)—N(R²⁰)(R²²), —N(R²⁰)—S(O)₂—R²⁰, carbocyclyl, aryl, heteroaryl or heterocyclyl; and wherein said carbocyclyl, aryl, heteroaryl or heterocyclyl are optionally substituted with one, two or three substituents independently selected from the group consisting of C₁₋₆ alkyl, C₂₋₄ alkynyl, halo, —NO₂, carbocyclyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰;

L is —O—, —S—, —C(O)—, —NHS(O)₂—, —S(O)₂NH—, —C(O)NH—, or —NHC(O)—; provided that when Y is —C(R¹¹)₂—, then R² is -L-R⁵, -L-C₁₋₆ alkylene-R⁵, —C₁₋₆ alkylene-L-R⁵ or —C₁₋₆ alkylene-L-C₁₋₆ alkylene-R⁵ and L is not —C(O)—; and when R² is -L-R⁵ or -L-C₁₋₆ alkylene-R⁵, then L is not —O—, —S—, —NHS(O)₂— or —NHC(O)—;

each R³ is independently hydrogen, deuterium, C₁₋₆ alkyl, carbocyclyl, aryl, heteroaryl or heterocyclyl; wherein said C₁₋₆ alkyl is optionally substituted with one, two or three substituents independently selected from the group consisting of halo, —NO₂, carbocyclyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰; wherein said carbocyclyl, aryl, heterocyclyl or heteroaryl are optionally further substituted with one, two or three substituents independently selected from the group consisting of halo, —NO₂, C₁₋₆ alkyl, aralkyl, carbocyclyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰; and wherein said C₁₋₆ alkyl, aralkyl, carbocyclyl, aryl, heterocyclyl or heteroaryl, are optionally further substituted with one, two or three substituents independently selected from the group consisting of halo, —NO₂, —N(R²⁰)(R²²), —C(O)—R²⁰, —C(O)—OR²⁰, —C(O)—N(R²⁰)(R²²), —CN and —O—R²⁰;

or when Y is —C(O)—, then R² and one of R³ can join together with the atoms to which they are attached to form a heterocyclyl; wherein said heterocyclyl is optionally substituted with one, two or three substituents independently selected from the group consisting of C₁₋₆ alkyl, —O—R²⁰, —N(R²⁰)(R²²), —N(R²⁰)—C(O)—OR²⁰ and —C(O)—OR²⁰; and wherein said C₁₋₆ alkyl is optionally substituted with one, two or three substituents independently selected from the group consisting of halo and heteroaryl;

each R⁴ is independently hydrogen, deuterium, C₁₋₆ alkyl, —C(O)—OR²⁶, —C(O)—N(R²⁶)(R²⁶), carbocyclyl, aryl, heteroaryl or heterocyclyl; wherein said C₁₋₆ alkyl is optionally substituted with one, two or three substituents independently selected from the group consisting of halo, —NO₂, carbocyclyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰; wherein said carbocyclyl, aryl, heterocyclyl or heteroaryl are optionally further substituted with one, two or three substituents independently selected from the group consisting of halo, —NO₂, C₁₋₆ alkyl, aralkyl, carbocyclyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰; and wherein said C₁₋₆ alkyl, aralkyl, carbocyclyl, aryl, heterocyclyl, heteroaryl, are optionally further substituted with one, two or three substituents independently selected from the group consisting of hydroxyl, halo, —NO₂, —N(R²⁰)(R²²), —C(O)—R²⁰, —C(O)—OR²⁰, —C(O)—N(R²⁰)(R²²), —CN and —O—R²⁰; or two R³ or two R⁴ together with the carbon atoms to which they are attached form an oxo;

R⁵ is carbocyclyl, aryl, heteroaryl or heterocyclyl; wherein said carbocyclyl, aryl, heteroaryl or heterocyclyl are optionally substituted with one, two or three substituents independently selected from the group consisting of C₁₋₆ alkyl, C₂₋₄ alkynyl, halo, —NO₂, carbocyclyl, aryl, heterocyclyl, heteroaryl, —N(R²⁰)(R²²), —N(R²⁰)—S(O)₂—R²⁰, —N(R²⁰)—C(O)—R²², —C(O)—R²⁰, —C(O)—OR²⁰, —C(O)—N(R²⁰)(R²²), —CN, oxo and —O—R²⁰; wherein said C₁₋₆ alkyl, carbocyclyl, aryl, heterocyclyl or heteroaryl are optionally further substituted with one, two or three substituents independently selected from the group consisting of halo, —NO₂, C₁₋₆ alkyl, carbocyclyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰; and wherein said C₁₋₆ alkyl, carbocyclyl, aryl, heterocyclyl or heteroaryl are optionally further substituted with one, two or three substituents independently selected from the group consisting of halo, aryl, —NO₂, —CF₃, —N(R²⁰)(R²²), —C(O)—R²⁰, —C(O)—OR²⁰, —C(O)—N(R²⁰)(R²²), —CN, —S(O)₂—R²⁰ and —O—R²⁰; R⁶ is hydrogen, C₁₋₆ alkyl or carbocyclyl; wherein said C₁₋₆ alkyl is optionally substituted with one, two or three substituents independently selected from the group consisting of halo, —NO₂, —N(R²⁰)(R²²), —C(O)—R²⁰, —C(O)—OR²⁰, —C(O)—N(R²⁰)(R²²), —CN and —O—R²⁰;

R⁷ is hydrogen, halo, —O—R or C₁₋₆ alkyl;

R¹¹ is hydrogen or C₁₋₄ alkyl;

R²⁰ and R²² are in each instance independently selected from the group consisting of hydrogen, C₁₋₆ alkyl, C₂₋₆ alkenyl, C₂₋₆ alkynyl, carbocyclyl, heterocyclyl, aryl and heteroaryl; wherein the C₁₋₆ alkyl, C₂₋₆ alkenyl, C₂₋₆ alkynyl, carbocyclyl, heterocyclyl, aryl and heteroaryl are optionally substituted with one, two or three substituents independently selected from the group consisting of hydroxyl, halo, C₁₋₄ alkyl, acylamino, oxo, —NO₂, —S(O)₂R²⁶, —CN, C₁₋₃ alkoxy, —CF₃, —OCF₃, —OCH₂CF₃, —C(O)—NH₂, aryl, carbocyclyl and heteroaryl; and wherein said heteroaryl is optionally further substituted with C₁₋₄ alkyl or carbocyclyl;

or when R²⁰ and R²² are attached to a common nitrogen atom, R²⁰ and R²² may join to form a heterocyclic or heteroaryl ring which is then optionally substituted with one, two or three substituents independently selected from the group consisting of hydroxyl, halo, C₁₋₄ alkyl, aralkyl, aryloxy, aralkyloxy, acylamino, —NO₂, —S(O)₂R²⁶, —CN, C₁₋₃ alkoxy, —CF₃, —OCF₃, aryl, heteroaryl and carbocyclyl; and

each R²⁶ is independently selected from the group consisting of hydrogen, C₁₋₄ alkyl, aryl and carbocyclyl; wherein the C₁₋₄ alkyl, aryl and carbocyclyl may be further substituted with from 1 to 3 substituents independently selected from the group consisting of hydroxyl, halo, C₁₋₄ alkoxy, —CF₃ and —OCF₃.

In some embodiments, Z¹ is CR⁷ (e.g., CH). In some embodiments, Z² is CR⁷ (e.g., CH). In some embodiments, each of Z¹ and Z² is independently CR⁷ (e.g., CH).

In some embodiments, Z³ is C-Q-R¹. In some embodiments, Q is a covalent bond. In some embodiments, R¹ is aryl (e.g., phenyl) optionally substituted with —O—R²⁰ (e.g., —OCF₃).

In some embodiments, Z⁴ is CR⁷ (e.g., CH).

In some embodiments, Y is —C(O)—. In some embodiments, X is —O—.

In some embodiments, R² is —C₁₋₆ alkylene-R⁵. In some embodiments, R² is —CH₂—R⁵.

In some embodiments, R⁵ is heteroaryl (e.g., a nitrogen-containing heteroaryl, e.g., pyrimidinyl).

In some embodiments, each R³ is independently hydrogen. In some embodiments, each R⁴ is independently hydrogen. In some embodiments, each R³ and R⁴ is independently hydrogen.

In some embodiments, when Y is —C(O)—, X is —O—, each R⁴ is hydrogen, R² and R³ together with the atoms to which they are attached form piperazinyl which is optionally substituted with tert-butoxycarbonyl and Q is a bond, then R¹ is not unsubstituted phenyl or morpholinyl. In some embodiments, when Y is —S(O)₂—, X is —O—, R² is benzyl, each R³ is hydrogen, Z⁴ is C-Q-R¹, Q is a bond and R¹ is aryl or heteroaryl, then both R⁴ are hydrogen.

In some embodiments, the compound of Formula (I) is a compound of Formula (I-a):

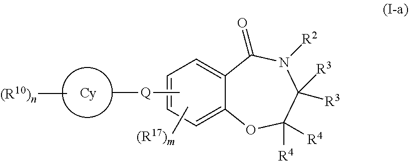

or a pharmaceutically acceptable salt, ester, stereoisomer, mixture of stereoisomers or tautomer thereof, wherein:

Cy is aryl, carbocyclyl, cycloalkenyl, heterocyclyl or heteroaryl;

Q is a covalent bond, —O—C₀₋₂ alkylene, —NR¹¹—C₀₋₂ alkylene, C₂ alkylene, C₂ alkenylene or C₂ alkynylene;

m is 0, 1, 2 or 3;

n is 0, 1, 2, 3, 4 or 5;

each R¹⁰ is independently selected from the group consisting of halo, —NO₂, —CN, —SF₅, —Si(CH₃)₃, —O—R²⁰, —S—R²⁰, —C(O)—R²⁰, —C(O)—OR²⁰, —N(R²⁰)(R²²), —C(O)—N(R²⁰)(R²²), —N(R²⁰)—C(O)—R²², —N(R²⁰)—C(O)—OR²², —N(R²⁰)—S(O)₂—R²⁶, —S(O)₂— R²⁰, —O—S(O)₂—R²⁰, —S(O)₂—N(R²⁰)(R²²), C₁₋₆ alkyl, C₂₋₄ alkenyl, C₂₋₄ alkynyl, carbocyclyl, aryl, heteroaryl and heterocyclyl; and wherein said C₁₋₆ alkyl, C₂₋₄ alkenyl, C₂₋₄ alkynyl, carbocyclyl, aryl, heteroaryl or heterocyclyl are optionally substituted with one, two or three substituents independently selected from the group consisting of halo, —NO₂, aryl, heterocyclyl, heteroaryl, C₁₋₆ alkyl, C₁₋₃ haloalkyl, carbocyclyl, —N(R²⁰)(R²²), —C(O)—R²⁰, —C(O)—OR²⁰, —C(O)—N(R²⁰)(R²²), —CN and —O—R²⁰;

R² is —C₁₋₆ alkylene-R⁵, -L-R⁵, -L-C₁₋₆ alkylene-R⁵, —C₁₋₆ alkylene-L-R⁵ or —C₁₋₆ alkylene-L-C₁₋₆ alkylene-R⁵; wherein each —C₁₋₆ alkyl ene is optionally substituted by one substituent independently selected from the group consisting of C₂₋₄ alkynyl, halo, —NO₂, —CN, —O—R²⁰, —N(R²⁰)(R²²), —C(O)—R²⁰, —C(O)—OR²⁶, —C(O)—N(R²⁰)(R²²), —N(R²⁰)—S(O)₂—R²⁰, carbocyclyl, aryl, heteroaryl or heterocyclyl; and wherein said carbocyclyl, aryl, heteroaryl or heterocyclyl are optionally substituted with one, two or three substituents independently selected from the group consisting of C₁₋₆ alkyl, C₂₋₄ alkynyl, halo, —NO₂, carbocyclyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰;

L is —O—, —S—, —C(O)—, —NHS(O)₂—, —S(O)₂NH—, —C(O)NH— or —NHC(O)—; provided that when R is -L-R or -L-C₁₋₆ alkylene-R, then L is not —O—, —S—, —NHS(O)₂— or —NHC(O)—; and

each R³ is independently hydrogen, deuterium, C₁₋₆ alkyl, carbocyclyl, aryl, heteroaryl or heterocyclyl; wherein said C₁₋₆ alkyl is optionally substituted with one, two or three substituents independently selected from the group consisting of halo, —NO₂, carbocyclyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰; wherein said carbocyclyl, aryl, heterocyclyl or heteroaryl are optionally further substituted with one, two or three substituents independently selected from the group consisting of halo, —NO₂, C₁₋₆ alkyl, aralkyl, carbocyclyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰; and wherein said C₁₋₆ alkyl, aralkyl, carbocyclyl, aryl, heterocyclyl or heteroaryl, are optionally further substituted with one, two or three substituents independently selected from the group consisting of halo, —NO₂, —N(R²⁰)(R²²), —C(O)—R²⁰, —C(O)—OR²⁰, —C(O)—N(R²⁰)(R²²), —CN and —O—R²⁰;

or R² and one of R³ can join together with the atoms to which they are attached to form a heterocyclyl; wherein said heterocyclyl is optionally substituted with one, two or three substituents independently selected from the group consisting of C₁₋₆ alkyl, —O—R²⁰, —N(R²⁰)(R²²), —N(R²⁰)—C(O)—OR²⁰ and —C(O)—OR²⁰; and wherein said C₁₋₆ alkyl is optionally substituted with one, two or three substituents independently selected from the group consisting of halo and heteroaryl;

each R⁴ is independently hydrogen, deuterium, C₁₋₆ alkyl, —C(O)—OR²⁶; —C(O)—N(R²⁶)(R²⁶), carbocyclyl, aryl, heteroaryl or heterocyclyl; wherein said C₁₋₆ alkyl is optionally substituted with one, two or three substituents independently selected from the group consisting of halo, —NO₂, carbocyclyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰; wherein said carbocyclyl, aryl, heterocyclyl or heteroaryl are optionally further substituted with one, two or three substituents independently selected from the group consisting of halo, —NO₂, C₁₋₆ alkyl, aralkyl, carbocyclyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰; and wherein said C₁₋₆ alkyl, aralkyl, carbocyclyl, aryl, heterocyclyl, heteroaryl, are optionally further substituted with one, two or three substituents independently selected from the group consisting of hydroxyl, halo, —NO₂, —N(R²⁰)(R²²), —C(O)—R²⁰, —C(O)—OR²⁰, —C(O)—N(R²⁰)(R²²), —CN and —O—R²⁰;

R⁵ is carbocyclyl, aryl, heteroaryl or heterocyclyl; wherein said carbocyclyl, aryl, heteroaryl or heterocyclyl are optionally substituted with one, two or three substituents independently selected from the group consisting of C₁₋₆ alkyl, C₂₋₄ alkynyl, halo, —NO₂, carbocyclyl, aryl, heterocyclyl, heteroaryl, —N(R²⁰)(R²²), —N(R²⁰)—S(O)₂—R²⁰, —N(R²⁰)—C(O)—R²², —C(O)—R²⁰, —C(O)—OR²⁰, —C(O)—N(R²⁰)(R²²), —CN, oxo and —O— wherein said C₁₋₆ alkyl, carbocyclyl, aryl, heterocyclyl or heteroaryl are optionally further substituted with one, two or three substituents independently selected from the group consisting of halo, —NO₂, C₁₋₆ alkyl, carbocyclyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰; and wherein said C₁₋₆ alkyl, carbocyclyl, aryl, heterocyclyl or heteroaryl are optionally further substituted with one, two or three substituents independently selected from the group consisting of halo, aryl, —NO₂, —CF₃, —N(R²⁰)(R²²), —C(O)—R²⁰, —C(O)—OR²⁰, —C(O)—N(R²⁰)(R²²), —CN, —S(O)₂—R²⁰ and —O—R²⁰;

R¹⁷ is halo, —O—R²⁰ or C₁₋₆ alkyl;

R²⁰ and R²² are in each instance independently selected from the group consisting of hydrogen, C₁₋₆ alkyl, C₂₋₆ alkenyl, C₂₋₆ alkynyl, carbocyclyl, heterocyclyl, aryl and heteroaryl; wherein the C₁₋₆ alkyl, C₂₋₆ alkenyl, C₂₋₆ alkynyl, carbocyclyl, heterocyclyl, aryl and heteroaryl are optionally substituted with one, two or three substituents independently selected from the group consisting of hydroxyl, halo, C₁₋₄ alkyl, acylamino, oxo, —NO₂, —S(O)₂R²⁶, —CN, C₁₋₃ alkoxy, —CF₃, —OCF₃, —OCH₂CF₃, —C(O)—NH₂, aryl, carbocyclyl and heteroaryl; and wherein said heteroaryl is optionally further substituted with C₁₋₄ alkyl or carbocyclyl; or when R²⁰ and R²² are attached to a common nitrogen atom, R²⁰ and R²² may join to form a heterocyclic or heteroaryl ring which is then optionally substituted with one, two or three substituents independently selected from the group consisting of hydroxyl, halo, C₁₋₄ alkyl, aralkyl, aryloxy, aralkyloxy, acylamino, —NO₂, —S(O)₂R²⁶, —CN, C₁₋₄ alkoxy, —CF₃, —OCF₃, aryl, heteroaryl and carbocyclyl; and each R²⁶ is independently selected from the group consisting of hydrogen, C₁₋₄ alkyl, aryl and carbocyclyl; wherein the C₁₋₄ alkyl, aryl and carbocyclyl may be further substituted with from 1 to 3 substituents independently selected from the group consisting of hydroxyl, halo, C₁₋₄ alkoxy, —CF₃ and —OCF₃.

In some embodiments, Q is a covalent bond.

In some embodiments, Cy is aryl (e.g., phenyl). In some embodiments, R¹⁰ is —O—R²⁰ (e.g., —OCF₃). In some embodiments, n is 1. In some embodiments, R¹⁰ is attached to the phenyl ring at the para position.

In some embodiments, m is 0.

In some embodiments, R² is —C₁₋₆ alkylene-R⁵. In some embodiments, R² is —CH₂—R⁵. In some embodiments, R⁵ is heteroaryl (e.g., a nitrogen-containing heteroaryl, e.g., pyrimidinyl).

In some embodiments, each R³ is independently hydrogen. In some embodiments, each R⁴ is independently hydrogen. In some embodiments, each R³ and R⁴ is independently hydrogen.

In some embodiments, when each R⁴ is hydrogen, R² and R³ together with the atoms to which they are attached form piperazinyl which is optionally substituted with tert-butoxycarbonyl, Q is a bond, and Cy is phenyl or morpholinyl, then n is 1, 2, 3, 4 or 5.

In some embodiments, the compound of Formula (I) is a compound of Formula (I-b):

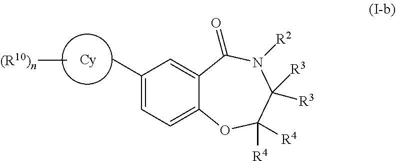

or a pharmaceutically acceptable salt, ester, stereoisomer, mixture of stereoisomers or tautomer thereof, wherein:

Cy is aryl, carbocyclyl, heterocyclyl or heteroaryl;

n is 0, 1, 2, 3, 4 or 5;

each R¹⁰ is independently selected from the group consisting of halo, —O—R²⁰, —S—R²⁰, —C(O)—R²⁰, —C(O)—OR²⁰, —N(R²⁰)(R²²), —C(O)—N(R²⁰)(R²2), —N(R²⁰)—C(O)—R²², —N(R²⁰)—C(O)—OR²², —N(R²⁰)—S(O)₂—R²⁶, —S(O)₂— R²⁰, —O—S(O)₂—R²⁰, —S(O)₂—N(R²⁰)(R²²), C₁₋₆ alkyl, C₂₋₄ alkenyl, C₂₋₄ alkynyl, carbocyclyl, aryl, heteroaryl and heterocyclyl; and wherein said C₁₋₆ alkyl, C₂₋₄ alkenyl, C₂₋₄ alkynyl, carbocyclyl, aryl, heteroaryl or heterocyclyl are optionally substituted with one, two or three substituents independently selected from the group consisting of halo, —NO₂, aryl, heterocyclyl, heteroaryl, C₁₋₆ alkyl, C₁₋₃ haloalkyl, carbocyclyl, —N(R²⁰)(R²²), —C(O)—R²⁰, —C(O)—OR²⁰, —C(O)—N(R²⁰)(R²²), —CN and —O—R²⁰;

R² is —C₁₋₆ alkylene-R⁵;

each R³ is independently hydrogen, deuterium, or C₁₋₆ alkyl;

each R⁴ is independently hydrogen, deuterium, C₁₋₆ alkyl;

R⁵ is carbocyclyl, aryl, heteroaryl or heterocyclyl; wherein said carbocyclyl, aryl, heteroaryl or heterocyclyl are optionally substituted with one, two or three substituents independently selected from the group consisting of C₁₋₆ alkyl, C₂₋₄ alkynyl, halo, —NO₂, carbocyclyl, aryl, heterocyclyl, heteroaryl, —N(R²⁰)(R²²), —N(R²⁰)—S(O)₂—R²⁰, —N(R²⁰)—C(O)—R²², —C(O)—R²⁰, —C(O)—OR²⁰, —C(O)—N(R²⁰)(R²²), —CN, oxo and —O— wherein said C₁₋₆ alkyl, carbocyclyl, aryl, heterocyclyl or heteroaryl are optionally further substituted with one, two or three substituents independently selected from the group consisting of halo, —NO₂, C₁₋₆ alkyl, carbocyclyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰; and wherein said C₁₋₆ alkyl, carbocyclyl, aryl, heterocyclyl or heteroaryl are optionally further substituted with one, two or three substituents independently selected from the group consisting of halo, aryl, —NO₂, —CF₃, —N(R²⁰)(R²²), —C(O)—R²⁰, —C(O)—OR²⁰, —C(O)—N(R²⁰)(R²²), —CN, —S(O)₂—R²⁰ and —O—R²⁰;

R²⁰ and R²² are in each instance independently selected from the group consisting of hydrogen, C₁₋₆ alkyl, C₂₋₆ alkenyl, C₂₋₆ alkynyl, carbocyclyl, heterocyclyl, aryl and heteroaryl; In some embodiments, Q is a covalent bond.

In some embodiments, Cy is aryl (e.g., phenyl). In some embodiments, R¹⁰ is —O—R²⁰ (e.g., —OCF₃). In some embodiments, n is 1.

In some embodiments, R² is —CH₂—R⁵. In some embodiments, R⁵ is heteroaryl (e.g., a nitrogen-containing heteroaryl, e.g., pyrimidinyl).

In some embodiments, each R³ is independently hydrogen. In some embodiments, each R⁴ is independently hydrogen. In some embodiments, each R³ and R⁴ is independently hydrogen.

In any and all aspects, in some embodiments, the compound of Formula (I) (e.g., Formula (I-a) or Formula (I-b)) is a compound selected from:
4-((3-methyloxetan-3-yl)methyl)-7-(4-(trifluoromethoxy)phenyl)-3,4-dihydrobenzo[f][1,4]oxazepin-5(2H)-one (1.);
4-(2-(pyrrolidin-1-yl)ethyl)-7-(4-(trifluoromethoxy)phenyl)-3,4-dihydrobenzo-[f][1,4]oxazepin-5(2H)-one (2.);
4-((5-cyclobutyl-1,3,4-oxadiazol-2-yl)methyl)-7-(4-(trifluoromethoxy)phenyl)-3,4-dihydrobenzo[f][1,4]oxazepin-5(2H)-one (3.);
4-((2,3-dihydrobenzo[b][1,4]dioxin-6-yl)methyl)-7-(4-(trifluoromethoxy)phenyl)-3 dihydrobenzo[f][1,4]oxazepin-5(2H)-one (4.);
4-(quinolin-2-ylmethyl)-7-(4-(trifluoromethoxy)phenyl)-3,4-dihydrobenzo-[f][1,4]-oxazepin-5(2H)-one (5.);
(R)-2-(pyrimidin-2-ylmethyl)-8-(4-(trifluoromethyl)phenyl)-3,4,12,12a-tetrahydro-1H-benzo[f]pyrazino[2,1-c][1,4]oxazepin-6(2H)-one (6.);
4-(cyclopropylmethyl)-7-(4-(trifluoromethoxy)phenyl)-3,4-dihydrobenzo[f][1,4]-oxazepin-5(2H)-one (7.);
(S)-3-methyl-4-(pyrimidin-2-ylmethyl)-7-(4-(trifluoromethyl)phenyl)-3,4-dihydrobenzo[f][1,4]oxazepin-5(2H)-one (8.);
(R)-3-methyl-4-(pyrimidin-2-ylmethyl)-7-(4-(trifluoromethyl)phenyl)-3,4-dihydrobenzo[f][1,4]oxazepin-5(2H)-one (9.);
6-((5-oxo-7-(4-(trifluoromethoxy)phenyl)-2,3-dihydrobenzo[f][1,4]oxazepin-4(5H)-yl)methyl)picolinonitrile (10.);
7-(4-(trifluoromethoxy)phenyl)-4-((6-(trifluoromethyl)pyridin-2-yl)methdihydrobenzo-[f][1,4]oxazepin-5(2H)-one (11.);
7-(4-(trifluoromethoxy)phenyl)-4-((6-(trifluoromethyl)pyridin-3-yl)methdihydrobenzo-[f][1,4]oxazepin-5(2H)-one (12.);
4-((6-methylpyridin-2-yl)methyl)-7-(4-(trifluoromethoxy)phenyl)-3,4 dihydrobenzo[f]-[1,4]-oxazepin-5(2H)-one (13.);
(2R, 11aS)-2-amino-7-(4-(trifluoromethyl)phenyl)-2,3,11,11a-tetrahydrobenzo-[f]pyrrolo[2,1-c][1,4]oxazepin-5(1H)-one (14.);
(R)-2-(2,2-difluoroethyl)-8-(4-(trifluoromethyl)phenyl)-3,4,12,12a-tetrahydro-1H-benzo[f]pyrazino-[2,1-c][1,4]oxazepin-6(2H)-one (15.);
(R)-2-ethyl-8-(4-(trifluoromethyl)phenyl)-3,4,12,12a-tetrahydro-1H-benzo[f]pyrazino-[2,1-c][1,4]oxazepin-6(2H)-one (16.);
(S)-2-(2,2-difluoroethyl)-8-(4-(trifluoromethyl)phenyl)-3,4,12,12a-tetrahydro-1H-benzo[f]pyrazino[2,1-c][1,4]oxazepin-6(2H)-one (17.);
(S)-2-ethyl-8-(4-(trifluoromethyl)phenyl)-3,4,12,12a-tetrahydro-1H-benzo[f]pyrazino[2,1-c][1,4]oxazepin-6(2H)-one (18.);
4-(pyrazin-2-ylmethyl)-7-(4-(trifluoromethoxy)phenyl)-3,4-dihydrobenzo[f]-[1,4]oxazepin-5(2H)-one (19.);
4-((5-methyloxazol-2-yl)methyl)-7-(4-(trifluoromethoxy)phenyl)-3,4-dihydrobenzo[f]-[1,4]oxazepin-5(2H)-one (20.);
7-(4-(trifluoromethoxy)phenyl)-4-(2-(2,5,5-trimethyl-1,3-dioxan-2-yl)ethyl)-3,4-dihydrobenzo[f][1,4]oxazepin-5(2H)-one (21.);
tert-butyl (2R,11aR)-5-oxo-7-(4-(trifluoromethyl)phenyl)-1,2,3,5,1 1,1 la-hexahydrobenzo[f]-pyrrolo[2,1-c][1,4]oxazepin-2-ylcarbamate (22.);
4-((5-(pyridin-2-yl)isoxazol-3-yl)methyl)-7-(4-(trifluoromethoxy)phenyl)-3,4-dihydrobenzo[f][1,4]oxazepin-5(2H)-one (23.);
4-((4,6-dimethoxypyrimidin-2-yl)methyl)-7-(4-(trifluoromethoxydihydrobenzo[f]-[1,4]oxazepin-5(2H)-one (24.);
ethyl 3-((5-oxo-7-(4-(trifluoromethoxy)phenyl)-2,3-dihydrobenzo[f][1,4]oxazepin-4(5H)-yl)methyl)benzoate (25.);
4-(2-(pyrimidin-2-yl)ethyl)-7-(4-(trifluoromethoxy)phenyl)-3,4-dihydrobenzo[f]-[1,4]oxazepin-5(2H)-one (26.);
4-(3,4-difluorobenzyl)-7-(4-(trifluoromethoxy)phenyl)-3,4-dihydrobenzo[f][1,4]oxazepin-5(2H)-one (27.);
4-(2-chlorobenzyl)-7-(4-(trifluoromethoxy)phenyl)-3,4-dihydrobenzo[f][1,4]oxazepin-5(2H)-one (28.);
4-(2,6-dichlorobenzyl)-7-(4-(trifluoromethoxy)phenyl)-3,4-dihydrobenzo[f]-[1,4]oxazepin-5(2H)-one (29.);
4-(2,6-difluorobenzyl)-7-(4-(trifluoromethyl)phenyl)-3,4-dihydrobenzo[f][1,4]oxazepin-5(2H)-one (30.);
4-(2-(1H-pyrazol-1-yl)ethyl)-7-(4-(trifluoromethoxy)phenyl)-3,4-dihydrobenzo[f][1,4]oxazepin-5(2H)-one (31.);
(2S, 11aS)-2-amino-7-(4-(trifluoromethyl)phenyl)-2,3,11,11a-tetrahydrobenzo[f]pyrrolo[2,1-c][1,4]oxazepin-5(1H)-one (32.);
4-(2-(pyridin-2-yl)ethyl)-7-(4-(trifluoromethoxy)phenyl)-3,4-dihydrobenzo[f]-[1,4]oxazepin-5(2H)-one (33.);
4-(2-fluorobenzyl)-7-(4-(trifluoromethoxy)phenyl)-3,4-dihydrobenzo[f][1,4]oxazepin-5(2H)-one (34.);
(R)-7-(4-(trifluoromethyl)phenyl)-2,3,11,11a-tetrahydrobenzo[f]pyrrolo[2,1-c][1,4]oxazepin-5(1H)-one (35.);
4-(pyrimidin-2-ylmethyl)-7-(4-(trifluoromethyl)phenyl)-3,4-dihydrobenzo[f]-[1,4]oxazepin-5(2H)-one (36.);
4-(4-fluorobenzyl)-7-(4-(trifluoromethoxy)phenyl)-3,4-dihydrobenzo[f][1,4]oxazepin-5(2H)-one (37.);
4-((1-methyl-1H-pyrazol-3-yl)methyl)-7-(4-(trifluoromethoxy)phenyl)-3,4-dihydrobenzo[f][1,4]oxazepin-5(2H)-one (38.);
4-((5-chloropyrimidin-2-yl)methyl)-7-(4-(trifluoromethoxy)phenyl)-3,4-dihydrobenzo[f]-[1,4]oxazepin-5(2H)-one (39.);
4-(pyridin-4-ylmethyl)-7-(4-(trifluoromethoxy)phenyl)-3,4-dihydrobenzo[f]-[1,4]oxazepin-5(2H)-one (40.);
4-((5-cyclopropyl-1,3,4-oxadiazol-2-yl)methyl)-7-(4-(trifluoromethoxy)phenyl)-3,4-dihydrobenzo[f][1,4]oxazepin-5(2H)-one (41.);
4-(2-(pyrimidin-2-yloxy)ethyl)-7-(4-(trifluoromethoxy)phenyl)-3,4-dihydrobenzo[f]-[1,4]oxazepin-5(2H)-one (42.);
4-(pyridin-3-ylmethyl)-7-(4-(trifluoromethoxy)phenyl)-3,4-dihydrobenzo[f]-[1,4]oxazepin-5(2H)-one (43.);
4-(pyridin-2-ylmethyl)-7-(4-(trifluoromethoxy)phenyl)-3,4-dihydrobenzo[f]-[1,4]oxazepin-5(2H)-one (44.);
4-(pyrimidin-2-ylmethyl)-7-(4-(trifluoromethoxy)phenyl)-3,4-dihydrobenzo[f][1,4]oxazepin-5(2H)-one (45.);
4-((3-methylpyridin-2-yl)methyl)-7-(4-(trifluoromethoxy)phenyl)-3,4-dihydrobenzo[f][1,4]oxazepin-5(2H)-one (46.);
(R)-2-(2,2,2-trifluoroethyl)-8-(4-(trifluoromethyl)phenyl)-3,4,12,12a-tetrahydro-1H-benzo[f]pyrazino[2,1-c][1,4]oxazepin-6(2H)-one (47.);
4-(pyrimidin-2-ylmethyl)-7-p-tolyl-3,4-dihydrobenzo[f][1,4]oxazepin-5(2H)-one (48.);
7-(4-chlorophenyl)-4-(pyrimidin-2-yl)_{3,4}-dihydrobenzo[f][1,4]oxazepin-5(2H)-one (49.);
7-(4-isopropylphenyl)-4-(pyrimidin-2-ylmethyl)-3,4-dihydrobenzo[f][1,4]oxazepin-5(2H)-one (50.);
7-(4-ethylphenyl)-4-(pyrimidin-2-ylmethyl)-3,4-dihydrobenzo[f][1,4]oxazepin-5 (2H)-one (51.);
7-(4-cyclopropylphenyl)-4-(pyrimidin-2-ylmethyl)-3,4-dihydrobenzo[f][1,4]oxazepin-5(2H)-one (52.);
(R)-4-(1-(pyrimidin-2-yl)ethyl)-7-(4-(trifluoromethyl)phenyl)-3,4-dihydrobenzo[f]-[1,4]oxazepin-5(2H)-one (53.);
7-(4-isobutoxyphenyl)-4-(pyrimidin-2-ylmethyl)-3,4-dihydrobenzo[f][1,4]oxazepin-5(2H)-one (54.);
7-(4-tert-butylphenyl)-4-(pyrimidin-2-ylmethyl)-3,4-dihydrobenzo[f][1,4]oxazepin-5(2H)-one (55.);
7-(4-cyclopropoxyphenyl)-4-(pyrimidin-2-ylmethyl)-3,4-dihydrobenzo[f][1,4]oxazepin-5(2H)-one (56.);
7-(4-fluorophenyl)-4-(pyrimidin-2-ylmethyl)-3,4-dihydrobenzo[f][1,4]oxazepin-5(2H)-one (57.);
7-(2-fluoro-4-(trifluoromethyl)phenyl)-4-(pyrimidin-2-ylmethyl)-3,4-dihydrobenzo[f]-[1,4]oxazepin-5(2H)-one (58.);
7-(3-fluoro-4-(2,2,2-trifluoroethoxy)phenyl)-4-(pyrimidin-2-ylmethyl)-3,4-dihydrobenzo[f]-[1,4]oxazepin-5(2H)-one (59.);
4-(pyrimidin-2-ylmethyl)-7-(4-(2,2,2-trifluoroethoxy)phenyl)-3,4-dihydrobenzo[f]-[1,4]oxazepin-5(2H)-one (60.);
7-(2-chloro-4-(trifluoromethyl)phenyl)-4-(pyrimidin-2-ylmethyl)-3,4-dihydrobenzo[f][1,4]oxazepin-5(2H)-one (61.);
7-(4-(trifluoromethoxy)phenyl)-4-((4-(trifluoromethyl)pyrimidin-2-yl)methyl)-3,4dihydrobenzo[f]-[1,4]oxazepin-5(2H)-one (62.);
7-(4-(trifluoromethoxy)phenyl)-4-((5-(6-(trifluoromethyl)pyridin-3-yl)-pyrimidin-2-yl)methyl)-3,4-dihydrobenzo[f][1,4]oxazepin-5(2H)-one (63.);
7-(4-chloro-2-fluorophenyl)-4-(pyrimidin-2-ylmethyl)-3,4-dihydrobenzo[f][1,4]oxazepin-5(2H)-one (64.);
1-(4-(5-oxo-4-(pyrimidin-2-ylmethyl)-2,3,4,5-tetrahydrobenzo[f][1,4]oxazepin-7-yl)phenyl)cyclopentanecarbonitrile (65.);
7-(4-ethoxyphenyl)-4-(pyrimidin-2-ylmethyl)-3,4-dihydrobenzo[f][1,4]oxazepin-5(2H)-one (66.);
7-(4-(difluoromethyl)phenyl)-4-(pyrimidin-2-ylmethyl)-3,4-dihydrobenzo[f]-[1,4]oxazepin-5(2H)-one (67.);
4-(imidazo[1,2-a]pyridin-2-ylmethyl)-7-(4-(trifluoromethyl)phenyl)-3,4-dihydrobenzo[f][1,4]oxazepin-5(2H)-one (68.);
4-((4-morpholinopyrimidin-2-yl)methyl)-7-(4-(trifluoromethoxy)phenyl)-3,4-dihydrobenzo[f][1,4]oxazepin-5(2H)-one (69.);
4-benzyl-7-(4-(trifluoromethoxy)phenyl)-3,4-dihydrobenzo[f][1,4]oxazepin-5 (2H)-one (70.);
4-(imidazo[1,2-a]pyridin-2-ylmethyl)-7-(4-(trifluoromethyl)phenyl)-3,4-dihydrobenzo[f][1,4]oxazepin-5(2H)-one (71.);
7-(3-fluoro-4-(trifluoromethyl)phenyl)-4-(pyrimidin-2-ylmethyl)-3,4-dihydrobenzo[f]-[1,4]oxazepin-5(2H)-one (72.);
4-((4-methoxypyrimidin-2-yl)methyl)-7-(4-(trifluoromethoxy)phenyl) dihydrobenzo[f]-[1,4]oxazepin-5(2H)-one (73.);
4-((4-methylpyrimidin-2-yl)methyl)-7-(4-(trifluoromethoxy)phenyl)-3,4-dihydrobenzo[f][1,4]oxazepin-5(2H)-one (74.);
4-((4-(piperidin-1-yl)pyrimidin-2-yl)methyl)-7-(4-(trifluoromethoxy)phenyl)-3,4-dihydrobenzo[f][1,4]oxazepin-5(2H)-one (75.);
4-((4-(dimethylamino)pyrimidin-2-yl)methyl)-7-(4-(trifluoromethoxy)phenyl)-3,4-dihydrobenzo[f][1,4]oxazepin-5(2H)-one (76.);
4-benzyl-7-(4-(trifluoromethyl)phenyl)-3,4-dihydrobenzo[f][1,4]oxazepin-5 (2H)-one (77.);
4-((3-methoxypyridin-2-yl)methyl)-7-(4-(trifluoromethoxy)phenyl)-3,4-dihydrobenzo[f]-[1,4]oxazepin-5(2H)-one (78.);
7-(4-(cyclobutylmethoxy)phenyl)-4-(pyrimidin-2-ylmethyl)-3,4-dihydrobenzo[f]-[1,4]oxazepin-5(2H)-one (79.);
7-(2-methyl-4-(trifluoromethyl)phenyl)-4-(pyrimidin-2-ylmethyl)-3,4-dihydrobenzo[f]-[1,4]oxazepin-5(2H)-one (80.);
7-(2-methyl-4-(trifluoromethoxy)phenyl)-4-(pyrimidin-2-ylmethyl)-3,4-dihydrobenzo[f]-[1,4]oxazepin-5(2H)-one (81.);
4-((1-(difluoromethyl)-1H-pyrazol-3-yl)methyl)-7-(4-(trifluoromethoxy)phenyl)-3,4-dihydrobenzo[f]-[1,4]oxazepin-5(2H)-one (82.);
7-(4-(trifluoromethoxy)phenyl)-4-((3-(trifluoromethyl)-1H-pyrazol-1-yl)methyl)-3,4-dihydrobenzo[f][1,4]oxazepin-5(2H)-one (83.);
4-(pyrimidin-2-ylmethyl)-7-(4-(2,2,2-trifluoroethyl)phenyl)-3,4-dihydrobenzo[f]-[1,4]oxazepin-5(2H)-one (84.);
4-(pyridin-2-ylmethyl)-7-(4-(2,2,2-trifluoroethyl)phenyl)-3,4-dihydrobenzo[f][1,4]oxazepin-5(2H)-one (85.);
4-((1-cyclopentyl-1H-pyrazol-3-yl)methyl)-7-(4-(trifluoromethoxy)phenyl)-3,4-dihydrobenzo[f][1,4]oxazepin-5(2H)-one (86.);
4-((1-ethyl-1H-pyrazol-3-yl)methyl)-7-(4-(trifluoromethoxy)phenyl)-3,4-dihydrobenzo[f][1,4]oxazepin-5(2H)-one (87.);
4-((1-methyl-1H-imidazol-4-yl)methyl)-7-(4-(trifluoromethoxy)phenyl)-3,4-dihydrobenzo[f][1,4]oxazepin-5(2H)-one (88.);
4-((4-methyl-1H-pyrazol-1-yl)methyl)-7-(4-(trifluoromethoxy)phenyl)-3,4-dihydrobenzo[f][1,4]oxazepin-5(2H)-one (89.);
4-((4-chloro-1H-pyrazol-1-yl)methyl)-7-(4-(trifluoromethoxy)phenyl)-3,4-dihydrobenzo[f][1,4]oxazepin-5(2H)-one (90.);
7-(4-(difluoromethyl)phenyl)-4-(pyridin-2-ylmethyl)-3,4-dihydrobenzo[f][1,4]oxazepin-5(2H)-one (91.);
7-(4-chloro-3-fluorophenyl)-4-(pyridin-2-ylmethyl)-3,4-dihydrobenzo[f][1,4]oxazepin-5(2H)-one (92.);
7-(4-(difluoromethoxy)phenyl)-4-(pyridin-2-ylmethyl)-3,4-dihydrobenzo[f][1,4]oxazepin-5(2H)-one (93.);
4-((1-methyl-1H-pyrazol-4-yl)methyl)-7-(4-(trifluoromethoxy)phenyl)-3,4-dihydrobenzo[f][1,4]oxazepin-5(2H)-one (94.);
4-(pyrimidin-2-ylmethyl)-7-(2,3,4-trifluorophenyl)-3,4-dihydrobenzo[f][1,4]oxazepin-5(2H)-one (95.);
7-(3,4-difluorophenyl)-4-(pyrimidin-2-ylmethyl)-3,4-dihydrobenzo[f][1,4]oxazepin-5(2H)-one (96.);
4-((3-fluoropyridin-2-yl)methyl)-7-(4-(trifluoromethoxy)phenyl)-3,4-dihydrobenzo[f]-[1,4]oxazepin-5(2H)-one (97.);
4-benzyl-9-fluoro-7-(4-(trifluoromethoxy)phenyl)-3,4-dihydrobenzo[f][1,4]oxazepin-5(2H)-one (98.);
4-benzyl-9-fluoro-7-(4-(trifluoromethyl)phenyl)-3,4-dihydrobenzo[f][1,4]oxazepin-5(2H)-one (99.);
4-benzyl-8-fluoro-7-(4-(trifluoromethoxy)phenyl)-3,4-dihydrobenzo[f][1,4]oxazepin-5(2H)-one (100.);
4-benzyl-8-fluoro-7-(4-(trifluoromethyl)phenyl)-3,4-dihydrobenzo[f][1,4]oxazepin-5(2H)-one (101.);
7-(4-chloro-3-fluorophenyl)-4-((3-fluoropyridin-2-yl)methyl)-3,4-dihydrobenzo[f][1,4]oxazepin-5(2H)-one (102.);
7-(2-fluoro-4-(trifluoromethyl)phenyl)-4-((3-fluoropyridin-2-yl)methyl)-3,4-dihydrobenzo[f][1,4]oxazepin-5(2H)-one (103.);
4-(5-oxo-4-(pyrimidin-2-ylmethyl)-2,3,4,5-tetrahydrobenzo[f][1,4]oxazepin-7-yl)phenyl trifluoromethanesulfonate (104.);
4-((5-methylpyrazin-2-yl)methyl)-7-(4-(trifluoromethoxy)phenyl)-3,4-dihydrobenzo[f][1,4]oxazepin-5(2H)-one (105.);
2,2,3,3-tetradeutero-4-(pyrimidin-2-ylmethyl)-7-(4-(trifluoromethoxy)phenyl)-3,4-dihydrobenzo[f][1,4]oxazepin-5(2H)-one (106.);
4-((6-methylpyrazin-2-yl)methyl)-7-(4-(trifluoromethoxy)phenyl)-3,4-dihydrobenzo[f]-[1,4]oxazepin-5(2H)-one (107.);
4-((3-fluoropyridin-2-yl)methyl)-7-(4-(trifluoromethyl)phenyl)-3,4-dihydrobenzo[f][1,4]oxazepin-5(2H)-one (108.);
N-(2-(5-oxo-7-(4-(trifluoromethoxy)phenyl)-2,3-dihydrobenzo[f][1,4]oxazepin-4(5H)-yl)ethyl)benzenesulfonamide (109.);
N-(2-(5-oxo-7-(4-(trifluoromethoxy)phenyl)-2,3-dihydrobenzo[f][1,4]oxazepin-4(5H)-yl)ethyl)cyclopropanesulfonamide (110.);
4-((1-methyl-1H-imidazol-2-yl)methyl)-7-(4-(trifluoromethoxy)phenyl)-3,4-dihydrobenzo[f][1,4]oxazepin-5(2H)-one (111.);
4-((1-benzyl-1H-imidazol-2-yl)methyl)-7-(4-(trifluoromethoxy)phenyl)-3,4-dihydrobenzo[f][1,4]oxazepin-5(2H)-one (112.);
4-(imidazo[1,2-a]pyridin-2-ylmethyl)-7-(4-(trifluoromethoxy)phenyl)-3,4-dihydrobenzo[f][1,4]oxazepin-5(2H)-one (113.);
N-cyclopropyl-3-(5-oxo-7-(4-(trifluoromethoxy)phenyl)-2,3-dihydrobenzo[f]-[1,4]oxazepin-4(5H)-yl)propane-1-sulfonamide (114.);
N-(2-(5-oxo-7-(4-(trifluoromethoxy)phenyl)-2,3-dihydrobenzo[f][1,4]oxazepin-4(5H)-yl)ethyl)pyrimidine-2-carboxamide (115.);
7-(4-(4-fluorophenoxy)phenyl)-4-(pyrimidin-2-ylmethyl)-3,4-dihydrobenzo[f]-[1,4]oxazepin-5(2H)-one (116.);
7-(4-phenoxyphenyl)-4-(pyrimidin-2-ylmethyl-3,4-dihydrobenzo[f][1,4]oxazepin-5(2H)-one (117.);
7-(3-phenoxyphenyl)-4-(pyrimidin-2-ylmethyl)-3,4-dihydrobenzo[f][1,4]oxazepin-5(2H)-one (118.);
7-(4-tert-butylcyclohex-1-enyl)-4-(pyrimidin-2-ylmethyl)-3,4-dihydrobenzo[f]-[1,4]oxazepin-5(2H)-one (119.);
7-cyclohexenyl-4-(pyrimidin-2-ylmethyl)-3,4-dihydrobenzo[f][1,4]oxazepin-5 (2H)-one (120.);
7-(4-methylcyclohex-1-enyl)-4-(pyrimidin-2-ylmethyl)-3,4-dihydrobenzo[f]-[1,4]oxazepin-5(2H)-one (121.);
7-(2-tert-butoxypyridin-4-yl)-4-(pyridin-2-ylmethyl)-3,4-dihydrobenzo[f][1,4]oxazepin-5(2H)-one (122.);
7-(1-methyl-2-oxo-1,2-dihydropyridin-4-yl)-4-(pyridin-2-ylmethyl)-3,4-dihydrobenzo[f][1,4]oxazepin-5(2H)-one (123.);
4-(pyridin-2-ylmethyl)-7-(5-(trifluoromethyl)pyridin-2-yl)-3,4-dihydrobenzo[f]-[1,4]oxazepin-5(2H)-one (124.);
7-(2-isopropylthiazol-4-yl)-4-(pyridin-2-ylmethyl)-3,4-dihydrobenzo[f][1,4]oxazepin-5(2H)-one (125.);
4-(pyridin-2-ylmethyl)-7-(5-(trifluoromethyl)thiophen-2-yl)-3,4-dihydrobenzo[f]-[1,4]oxazepin-5(2H)-one (126.);
7-(5-cyclopropylthiophen-2-yl)-4-(pyridin-2-ylmethyl)-3,4-dihydrobenzo[f]-[1,4]oxazepin-5(2H)-one (127.);
7-(5-cyclopropylthiophen-2-yl)-4-(pyrimidin-2-ylmethyl)-3,4-dihydrobenzo[f-][1,4]oxazepin-5(2H)-one (128.);
4-(pyrimidin-2-ylmethyl)-7-(5-(trifluoromethyl)thiophen-2-yl)-3,4-dihydrobenzo[f]-[1,4]oxazepin-5(2H)-one (129.);
4-(pyrimidin-2-ylmethyl)-7-((4-(trifluoromethoxy)phenyl)ethynyl)-3,4-dihydrobenzo[f][1,4]oxazepin-5(2H)-one (130.);
7-(phenylethynyl)-4-(pyrimidin-2-ylmethyl)-3,4-dihydrobenzo[f][1,4]oxazilone (131.);
4-(pyrimidin-2-ylmethyl)-7-((4-(trifluoromethyl)phenyl)ethynyl)-3,4-dihydrobenzo[f]-[1,4]oxazepin-5(2H)-one (132.);
4-(pyridin-2-ylmethyl)-7-(4-(trifluoromethyl)phenethyl)-3,4-dihydrobenzo[f]-[1,4]oxazepin-5(2H)-one (133.);
4-(pyrimidin-2-ylmethyl)-7-(4-(1xifluoromethyl)phenethyl)-3,4-dihydrobenzo[f]-[1,4]oxazepin-5(2H)-one (134.);
4-((3-fluoropyridin-2-yl)methyl)-7-(4-(trifluoromethyl)phenethyl)-3,4-dihydrobenzo[f][1,4]oxazepin-5(2H)-one (135.);
(E)-4-benzyl-7-(4-(1 xifluoromethyl)styryl)-3,4-dihydrobenzo[f][1,4]oxazepin-5(2H)-one (136.);
4-benzyl-7-(4-(trifluoromethyl)phenethyl)-3,4-dihydrobenzo[f][1,4]oxazepin-5 (2H)-one (137.);
2-((pyrimidin-2-yl)methyl)-8-(4-(trifluoromethyl)phenyl)-3,4-dihydro-2H-benzo[b][1,4,5]oxathiazepin-sulfone (138.);
2-((5-chloropyrimidin-2-yl)methyl)-8-(4-(trifluoromethyl)phenyl)-3,4-dihydro-2H-benzo[b][1,4,5]oxathiazepin-sulfone (139.);
4-(2-(benzyloxy)ethyl)-1-methyl-7-(4-(trifluoromethoxy)phenyl)-3,4-dihydro-1H-benzo[e][1,4]diazepine-2,5-dione (140.);
4-benzyl-7-(4-(trifluoromethyl)phenyl)-3,4-dihydro-1H-benzo[e][1,4]diazepine-2,5-dione (141.);
4-benzyl-1-methyl-7-(4-(trifluoromethyl)phenyl)-3,4-dihydro-1H-benzo[e][1,4]diazepine-2,5-dione (142.);
5-benzyl-8-(4-(trifluoromethyl)phenyl)-4H-benzo[f]imidazo[1,2-a][1,4]diazepin-6(5H)-one (143.);
4-benzyl-7-(4-(trifluoromethoxy)phenyl)_{3,4}-dihydropyrido[4,3-f][1,4]oxazepin-5(2H)-one (144.);
4-benzyl-7-(4-(trifluoromethoxy)phenyl)-3,4-dihydropyrido[2,3-f][1,4]oxazepin-5(2H)-one (145.);
4-benzyl-7-(4-(trifluoromethyl)phenyl)-3,4-dihydropyrido[2,3-f][1,4]oxazepin-5(2H)-one (146.);
4-(pyrimidin-2-ylmethyl)-7-(4-(trifluoromethoxy)phenyl)-3,4-dihydropyrido[f]-[1,4]oxazepin-5(2H)-one (147.);
4-((4-methylpyrimidin-2-yl)methyl)-7-(4-(trifluoromethoxy)phenyl)-3,4-dihydropyrido[4,3-f][1,4]oxazepin-5(2H)-one (148.);
4-(cyclopropylmethyl)-7-(4-(trifluoromethoxy)phenyl)-3,4-dihydropyrido[4,3-f][1,4]oxazepin-5(2H)-one (149.);
4-((3-methoxypyridin-2-yl)methyl)-7-(4-(trifluoromethoxy)phenyl)-3,4-dihydropyrido[4,3-f][1,4]oxazepin-5(2H)-one (150.);
4-((3-fluoropyridin-2-yl)methyl)-7-(4-(trifluoromethoxy)phenyl)-3,4-dihydropyrido[4,3-f][1,4]oxazepin-5(2H)-one (151.);
4-((4-methoxypyrimidin-2-yl)methyl)-7-(4-(trifluoromethoxy)phenyl)-3,4-dihydropyrido[4,3-f][1,4]oxazepin-5(2H)-one (152.);
4-(pyrimidin-2-ylmethyl)-7-(4-(trifluoromethoxy)phenylamino)-3,4-dihydrobenzo[f]-[1,4]oxazepin-5(2H)-one (153.);
4-(pyrimidin-2-ylmethyl)-7-(4-(trifluoromethoxy)phenoxy)-3,4-dihydrobenzo[f]-[1,4]oxazepin-5(2H)-one (154.);
4-(pyrimidin-2-ylmethyl)-7-(4-(trifluoromethyl)phenylamino)-3,4-dihydrobenzo[f]-[1,4]oxazepin-5(2H)-one (155.);
4-(pyridin-2-ylmethyl)-7-(4-(trifluoromethoxy)phenylamino)-3,4-dihydrobenzo[f]-[1,4]oxazepin-5(2H)-one (156.);
4-(pyridin-2-ylmethyl)-7-(4-(trifluoromethyl)phenylamino)-3,4-dihydrobenzo[f][1,4]oxazepin-5(2H)-one (157.);
7-(methyl(4-(trifluoromethoxy)phenyl)amino)-4-(pyrimidin-2-ylmethyl)-3,4-dihydrobenzo[f][1,4]oxazepin-5(2H)-one (158.);
4-(2,2-difluoroethyl)-7-(4-(trifluoromethoxy)phenyl)-3,4-dihydrobenzo[f][1,4]oxazepin-5(2H)-one (159.);
4-(2-methoxyethyl)-7-(4-(trifluoromethoxy)phenyl)-3,4-dihydrobenzo[f][1,4]oxazepin-5(2H)-one;
(R)-3-methyl-7-(4-(trifluoromethyl)phenyl)-3,4-dihydrobenzo[f][1,4]oxazepin-5 (2H)-one (160.);
4-methyl-7-(4-(trifluoromethoxy)phenyl)-3,4-dihydrobenzo[f][1,4]oxazepin-5 (2H)-one (161.);
(S)-3-isopropyl-7-(4-(trifluoromethyl)phenyl)-3,4-dihydrobenzo[f][1,4]oxazepin-5(2H)-one (162.);
3-(pyridin-2-ylmethyl)-7-(4-(trifluoromethoxy)phenyl)-3,4-dihydrobenzo[f]-[1,4]oxazepin-5(2H)-one (163.);
N-(2-(5-oxo-7-(4-(trifluoromethoxy)phenyl)-2,3-dihydrobenzo[f][1,4]oxazepin-4(5H)-yl)ethyl)ethanesulfonamide (164.);
4-(3-(azetidin-1-ylsulfonyl)propyl)-7-(4-(trifluoromethoxy)phenyl)-3,4-dihydrobenzo[f][1,4]oxazepin-5(2H)-one (165.);
(4-(trifluoromethoxy)phenyl)-2,3-dihydrobenzo[f][1,4]oxazepin-4(5H)-yl)methanone (166.);
phenyl(7-(4-(trifluoromethoxy)phenyl)-2,3-dihydrobenzo[f][1,4]oxazepin-4(5H)-yl)methanone (167.);
(1-methylcyclopropyl)(7-(4-(trifluoromethoxy)phenyl)-2,3-dihydrobenzo[f][1,4]oxazepin-4(5H)-yl)methanone (168.);
(3,3-difluorocyclobutyl)(7-(4-(trifluoromethoxy)phenyl)-2,3-dihydrobenzo[f]-[1,4]oxazepin-4(5H)-yl)methanone (169.);
(1-methyl-1H-pyrazol-4-yl)(7-(4-(trifluoromethoxy)phenyl)-2,3-dihydrobenzo[f]-[1,4]oxazepin-4(5H)-yl)methanone (170.);
(1H-pyrazol-3-yl)(7-(4-(trifluoromethoxy)phenyl)-2,3-dihydrobenzo[f][1,4]oxazepin-4(5H)-yl)methanone (171.);
pyrazin-2-yl(7-(4-(trifluoromethoxy)phenyl)-2,3-dihydrobenzo[f][1,4]oxazepin-4(5H)-yl)methanone (172.);
pyridazin-3-yl(7-(4-(trifluoromethoxy)phenyl)-2,3-dihydrobenzo[f][1,4]oxazepin-4(5H)-yl)methanone (173.);
2-(pyridin-2-yl)-1-(7-(4-(trifluoromethyl)phenyl)-2,3-dihydrobenzo[f][1,4]oxazepin-4(5H)-yl)ethanone (174.);
2-(pyrimidin-2-yl)-1-(7-(4-(trifluoromethyl)phenyl)-2,3-dihydrobenzo[f][1,4]oxazepin-4(5H)-yl)ethanone (175.);
(1-methyl-1H-imidazol-5-yl)(7-(4-(trifluoromethyl)phenyl)-2,3-dihydrobenzo[f]-[1,4]oxazepin-4(5H)-yl)methanone (176.);
(1H-imidazol-2-yl)(7-(4-(trifluoromethyl)phenyl)-2,3-dihydrobenzo[f][1,4]oxazepin-4(5H)-yl)methanone (177.);
(1-methyl-1H-imidazol-2-yl)(7-(4-(trifluoromethyl)phenyl)-2,3-dihydrobenzo[f]-[1,4]oxazepin-4(5H)-yl)methanone (178.);
(R)-(2-methyl-7-(4-(trifluoromethoxy)phenyl)-2,3-dihydrobenzo[f][1,4]oxazepin-4(5H)-yl)(pyrimidin-2-yl)methanone (179.);
tert-butyl 2-(7-(4-(trifluoromethyl)phenyl)-2,3,4,5-tetrahydrobenzo[f][1,4]oxazepine-4-carbonyl)-5,6-dihydroimidazo[1,2-a]pyrazine-7(8H)-carboxylate (180.);
(1H-1,2,4-triazol-3-yl)(7-(4-(trifluoromethyl-4(5H)-yl)methanone (181.);
(1,5-dimethyl-1H-pyrazol-3-yl)(7-(4-(trifluoromethoxy)phenyl)-2,3-dihydrobenzo[f]-[1,4]oxazepin-4(5H)-yl)methanone (182.);

or a pharmaceutically acceptable salt, ester, stereoisomer, mixture of stereoisomers or tautomer thereof.

In some embodiments, the compound of Formula (I) is 4-(pyrimidin-2-ylmethyl)-7-(4-(trifluoromethoxy)phenyl)-3,4-dihydrobenzo[f][1,4]oxazepin-5(2H)-one (i.e., eleclazine, GS-6615, CAS 1443211-72-0), or a pharmaceutically acceptable salt, ester, stereoisomer, mixture of stereoisomers or tautomer thereof. In some embodiments, the compound of Formula (I) is

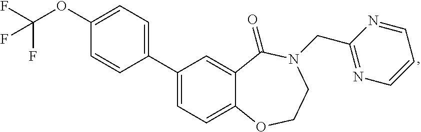 [SMILES: O=C1c2cc(-c3ccc(OC(F)(F)F)cc3)ccc2OCCN1Cc1ncccn1]

or a pharmaceutically acceptable salt, ester, stereoisomer, mixture of stereoisomers or tautomer thereof. In some embodiments, the compound of Formula (I) is disclosed in International Patent Publication No. WO 2013/0064585, International International Patent Publication No. WO 2014/179492, International International Patent Publication No. WO 2015/123434, and International Patent Publication No. WO 2015/123519, each of which is incorporated herein by reference in its entirety.

### Combination Therapy

A compound or composition described herein (e.g., for use in modulating a sodium ion channel, e.g., the late sodium (INaL) current) may be administered in combination with another agent or therapy. A subject to be administered a compound disclosed herein may have a disease, disorder, or condition, or a symptom thereof, that would benefit from treatment with another agent or therapy. These diseases or conditions can relate to epilepsy or an epilepsy syndrome, a neurodevelopmental disorder, pain, migraine, headache, a neuromuscular disorder, or a psychiatric disorder (e.g., bipolar disorder).

### Antiepilepsy Agents

Anti-epilepsy agents include brivaracetam, cannabidiol, carbamazepine, clobazam, clonazepam, diazepam, divalproex, eslicarbazepine, ethosuximide, ezogabine, fenfluramine, felbamate, gabapentin, lacosamide, lamotrigine, levetiracetam, lorazepam, neuroactive steroids, oxcarbezepine, permpanel, phenobarbital, phenytoin, pregabalin, primidone, rufinamide, tigabine, topiramate, valproic acid, vigabatrin, zonisamide.

### Antimigraine Agents

Anti-migraine agents include ergots, triptans (sumatriptan, frovatriptan), beta-blockers, tricylic antipressants (amitryptyline), topiramate, and CGRP inhibitors.

Accordingly, one aspect of the invention provides for a composition comprising the sodium channel blockers of the invention and at least one therapeutic agent. In an alternative embodiment, the composition comprises the sodium channel blockers of the invention and at least two therapeutic agents. In further alternative embodiments, the composition comprises the sodium channel blockers of the invention and at least three therapeutic agents, the sodium channel blockers of the invention and at least four therapeutic agents, or the sodium channel blockers of the invention and at least five therapeutic agents.

The methods of combination therapy include co-administration of a single formulation containing the sodium channel blockers of the invention and therapeutic agent or agents, essentially contemporaneous administration of more than one formulation comprising the sodium channel blocker of the invention and therapeutic agent or agents, and consecutive administration of a sodium channel blocker of the invention and therapeutic agent or agents, in any order, wherein preferably there is a time period where the sodium channel blocker of the invention and therapeutic agent or agents simultaneously exert their therapeutic effect.

### EXAMPLE

### Example 1 the Effect of Eleclazine on Human Nav1.2 Channel Variants

### Cell Culture and Transfections

Chinese hamster ovary (CHO) cells were cultured in Dulbecco's Modified Eagle Medium: Nutrient Mixture F-12 (Thermo Fisher Scientific, Waltham, Mass.) supplemented with 10% (v/v) fetal bovine serum (Thermo Fisher Scientific) and 50 IU/ml penicillin (Thermo Fisher Scientific) at 37° C. with 5% CO₂. The cells were grown in T25 cm² flasks (BD Biosciences, San Jose, Calif., USA) to ˜80% confluency, and then transiently co-transfected with wild-type or mutant pcDNA3.1(+)-Nav1.2 (4 μg) and enhanced green fluorescent protein (eGFP; 1 ag; Clontech, Mountain View, Calif.), using Lipofectamine 3000 Reagent (Thermo Fisher Scientific). After transfections, cells were incubated at 37° C. in 5% CO₂. 24 hours post-transfection, the cells were detached using TrypLE Express Reagent (Thermo Fisher Scientific), plated on 13 mm diameter glass coverslips (Menzel-Glaser, Thermo Fisher Scientific), and incubated at 30° C. in 5% CO₂. Electrophysiological recordings were performed 48 to 72 hours post transfection.

### Electrophysiology

Depolarization-activated whole-cell sodium currents (I_{Na}) from CHO cells transiently expressing wild-type or mutant Nav1.2 channels were recorded using an Axopatch 200B amplifier (Molecular Devices, Sunnyvale, Calif.) controlled by a pCLAMP 9/DigiData 1440 acquisition system (Molecular Devices). Currents and potentials were low-pass filtered at 10 kHz and digitized at 50 kHz. Data were analysed off-line using Clampfit 9.2 (Molecular Devices) and Origin 9.0 (Microcal Software Inc., Northampton, Mass.).

Experiments were performed at room temperature (23±0.5° C.). Cells were placed into a ˜0.1 ml recording chamber and superfused with extracellular solution at a constant rate of ˜1 ml/min. The extracellular bath solution contained 145 mM NaCl, 5 mM CsCl, 2 mM CaCl₂, 1 mM MgCl₂, 5 mM glucose, 5 mM sucrose, 10 mM Hepes (pH=7.4 with NaOH and osmolarity of ˜320 mosmol l⁻¹), whereas the intracellular solution contained 5 mM CsCl, 120 mM CsF, 10 mM NaCl, 11 mM EGTA, 1 mM CaCl₂, 1 mM MgCl₂, 2 mM Na₂ATP, 10 mM Hepes (pH=7.3 with CsOH and osmolarity of ˜300 mosmol l⁻¹).

Patch electrodes were pulled from borosilicate glass capillaries (GC150TF-7.5, Harvard Apparatus Ltd.) and typically exhibited resistance values of 1.2-1.5 MQ. Liquid junction potentials were corrected and series resistance values, typically of 2-2.5M, were 85-90% compensated. To minimize possible voltage errors, relatively small CHO cells of 12-20 pF cell capacitance (Cₘ=15.9+1.3 pF, n=19) were selected. The leak and capacitive currents were corrected using a −P/4 pulse protocol.

Once whole-cell recording configuration was established, the current-voltage (I-V) relationships were determined from a holding potential (HP) of −120 mV. I_{Na} were elicited by 40-ms depolarizing voltage steps, applied in the voltage range between −80 and +60 mV at 1 Hz. Peak I_{Na} was defined as the difference between peak and steady-state currents recorded during depolarization and when keeping the cell at HP, respectively. The voltage step resulting in the largest inward peak INa was chosen as step stimulus (depolarizing pulse) for the protocol to evaluate the effect of eleclazine. Experiments were only commenced when the step stimulus-elicited peak INa amplitude showed less than 5% change (usually increase) within a 5-min recording period. At the end of most experiments, 200 nM tetrodotoxin was applied to the block the sodium current (INa). Late (persistent) INa (INaL) was determined at 100 ms after the onset of the step voltage command by either subtracting traces recorded in the presence of tetrodotoxin from control, or by estimating inward INa after −P/4 leak correction. Both methods resulted in similar values and for comprehensive mutant and wild-type data analysis the −P/4 method was used. To enhance INaL through wild-type Nav1.2 channels sea anemone toxin (3 nM ATX-II) was used. Various concentrations of eleclazine in extracellular solution were prepared from 10 mM eleclazine stock solution. Concentration-response relationships were determined applying at least three incremental concentrations of eleclazine to individual cells.

### Chemicals

ATX-II and tetrodotoxin were purchased from Tocris Bioscience (Bristol, UK) and Alomone Laboratories (Israel). ATX-II was dissolved in de-ionized water to a stock concentration of 20 μM, whereas tetrodotoxin was dissolved in de-ionized water to a stock concentration of 10 μM. Eleclazine was dissolved in sterile dimethyl sulfoxide (DMSO) to a stock concentration of 10 mM. The aliquots of all above solutions were stored at −20° C. and added on the day of the experiment to the bath solution.

### Curve Fitting and Statistical Analysis

Data were analysed off-line using Clampfit 9.2 (Molecular Devices) and Origin 2017 (Microcal Software Inc., Northampton, Mass.). Current amplitudes obtained in the presence of a eleclazine (I) were normalized to current amplitudes obtained under control conditions (I₀). Concentration-response data were obtained by plotting averaged relative peak current amplitude (I/I₀) as function of eleclazine concentration ([eleclazine]). The half-maximal inhibitory concentration (IC₅₀) values were determined after fitting the Hill equation I=I₀{[eleclazine]^{h}/(IC₅₀^{h}+[eleclazine]^{h})} to concentration-response data, where h is the Hill coefficient (slope). Data are shown as mean±SEM; n, number of experiments.

Table 1 shows the IC₅₀ values and Hill slope values of eleclazine for the normalized peak I_{Na} and late I_{Na} in WT and mutant cell lines.

TABLE 1
Half-maximal inhibitory concentrations of eleclazine
in CHO cells transiently expressing human wild-type (WT)
Naᵥ1.2, R1882Q Naᵥ1.2, or K905N Naᵥ1.2channels.

| Naᵥ1.2 channel | IC₅₀ (μM) | Hill slope | | IC₅₀ (μM) | Hill slope | |
|---|---|---|---|---|---|---|
| variant | Peak I_{Na} | Peak I_{Na} | n | Late I_{Na} | Late I_{Na} | n |
| WT | 180 ± 8.2 | 0.63 ± 0.07 | 4 | 0.99 ± 0.09 | 0.94 ± 0.06 | 4 |
| R1882Q | 456 ± 13 | 0.36 ± 0.14 | 7 | 1.06 ± 0.6 | 0.64 ± 0.2 | 5 |
| K905N | 1700 ± 60 | 0.78 ± 0.2 | 5 | 0.69 ± 0.1 | 0.55 ± 0.06 | 4 |

IC₅₀ and Hill slope values were obtained after fitting the Hill equation to concentration-response data (see details of the fitting procedure in the Materials and Methods section). Data are mean±SEM; n, number of experiments. Each data point was obtained from 4 to 7 individual experiments.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A method of treating a neurodevelopmental disorder, wherein the method comprises administering to a subject in need thereof a compound of Formula (I-a): 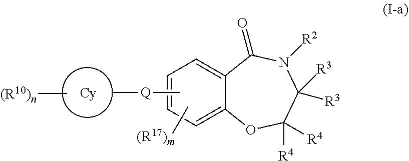
or a pharmaceutically acceptable salt, ester, stereoisomer, mixture of stereoisomers or tautomer thereof, wherein:
Cy is aryl, carbocyclyl, cycloalkenyl, heterocyclyl or heteroaryl;
Q is a covalent bond, —O—C₀₋₂ alkylene, —NR¹¹—C₀₋₂ alkylene, C₂ alkylene, C₂ alkenylene or C₂ alkynylene;
m is 0, 1, 2 or 3;
n is 0, 1, 2, 3, 4 or 5;
each R¹⁰ is independently selected from the group consisting of halo, —NO₂, —CN, —SF₅, —Si(CH₃)₃, —O—R²⁰, —S—R²⁰, —C(O)—R²⁰, —C(O)—OR²⁰, —N(R²⁰)(R²²), —C(O)—N(R²⁰)(R²²), —N(R²⁰)—C(O)—R²², —N(R²⁰)—C(O)—OR²², —N(R²⁰)—S(O)₂—R²⁶, —S(O)₂— R²⁰, —O—S(O)₂—R²⁰, —S(O)₂—N(R²⁰)(R²²), C₁₋₆ alkyl, C₂₋₄ alkenyl, C₂₋₄ alkynyl, carbocyclyl, aryl, heteroaryl and heterocyclyl; and wherein said C₁₋₆ alkyl, C₂₋₄ alkenyl, C₂₋₄ alkynyl, carbocyclyl, aryl, heteroaryl or heterocyclyl are optionally substituted with one, two or three substituents independently selected from the group consisting of halo, —NO₂, aryl, heterocyclyl, heteroaryl, C₁₋₆ alkyl, C₁₋₃ haloalkyl, carbocyclyl, —N(R²⁰)(R²²), —C(O)—R²⁰, —C(O)—OR²⁰, —C(O)—N(R²⁰)(R²²), —CN and —O—R²⁰;
R² is —C₁₋₆ alkylene-R⁵, -L-R⁵, -L-C₁₋₆ alkylene-R⁵, —C₁₋₆ alkylene-L-R⁵ or —C₁₋₆ alkylene-L-C₁₋₆ alkylene-R⁵; wherein each —C₁₋₆ alkyl ene is optionally substituted by one substituent independently selected from the group consisting of C₂₋₄ alkynyl, halo, —NO₂, —CN, —O—R²⁰, —N(R²⁰)(R²²), —C(O)—R²⁰, —C(O)—OR²⁶, —C(O)—N(R²⁰)(R²²), —N(R²⁰)—S(O)₂—R²⁰, carbocyclyl, aryl, heteroaryl or heterocyclyl; and wherein said carbocyclyl, aryl, heteroaryl or heterocyclyl are optionally substituted with one, two or three substituents independently selected from the group consisting of C₁₋₆ alkyl, C₂₋₄ alkynyl, halo, —NO₂, carbocyclyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰;
L is —O—, —S—, —C(O)—, —NHS(O)₂—, —S(O)₂NH—, —C(O)NH— or —NHC(O)—; provided that when R is -L-R or -L-C₁₋₆ alkylene-R, then L is not —O—, —S—, —NHS(O)₂— or —NHC(O)—; and
each R³ is independently hydrogen, deuterium, C₁₋₆ alkyl, carbocyclyl, aryl, heteroaryl or heterocyclyl; wherein said C₁₋₆ alkyl is optionally substituted with one, two or three substituents independently selected from the group consisting of halo, —NO₂, carbocyclyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰; wherein said carbocyclyl, aryl, heterocyclyl or heteroaryl are optionally further substituted with one, two or three substituents independently selected from the group consisting of halo, —NO₂, C₁₋₆ alkyl, aralkyl, carbocyclyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰; and wherein said C₁₋₆ alkyl, aralkyl, carbocyclyl, aryl, heterocyclyl or heteroaryl, are optionally further substituted with one, two or three substituents independently selected from the group consisting of halo, —NO₂, —N(R²⁰)(R²²), —C(O)—R²⁰, —C(O)—OR²⁰, —C(O)—N(R²⁰)(R²²), —CN and —O—R²⁰;
or R² and one of R³ can join together with the atoms to which they are attached to form a heterocyclyl; wherein said heterocyclyl is optionally substituted with one, two or three substituents independently selected from the group consisting of C₁₋₆ alkyl, —O—R²⁰, —N(R²⁰)(R²²), —N(R²⁰)—C(O)—OR²⁰ and —C(O)—OR²⁰; and wherein said C₁₋₆ alkyl is optionally substituted with one, two or three substituents independently selected from the group consisting of halo and heteroaryl;
each R⁴ is independently hydrogen, deuterium, C₁₋₆ alkyl, —C(O)—OR²⁶; —C(O)—N(R²⁶)(R²⁶), carbocyclyl, aryl, heteroaryl or heterocyclyl; wherein said C₁₋₆ alkyl is optionally substituted with one, two or three substituents independently selected from the group consisting of halo, —NO₂, carbocyclyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰; wherein said carbocyclyl, aryl, heterocyclyl or heteroaryl are optionally further substituted with one, two or three substituents independently selected from the group consisting of halo, —NO₂, C₁₋₆ alkyl, aralkyl, carbocyclyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰; and wherein said C₁₋₆ alkyl, aralkyl, carbocyclyl, aryl, heterocyclyl, heteroaryl, are optionally further substituted with one, two or three substituents independently selected from the group consisting of hydroxyl, halo, —NO₂, —N(R²⁰)(R²²), —C(O)—R²⁰, —C(O)—OR²⁰, —C(O)—N(R²⁰)(R²²), —CN and —O—R²⁰;
R⁵ is carbocyclyl, aryl, heteroaryl or heterocyclyl; wherein said carbocyclyl, aryl, heteroaryl or heterocyclyl are optionally substituted with one, two or three substituents independently selected from the group consisting of C₁₋₆ alkyl, C₂₋₄ alkynyl, halo, —NO₂, carbocyclyl, aryl, heterocyclyl, heteroaryl, —N(R²⁰)(R²²), —N(R²⁰)—S(O)₂—R²⁰, —N(R²⁰)—C(O)—R²², —C(O)—R²⁰, —C(O)—OR²⁰, —C(O)—N(R²⁰)(R²²), —CN, oxo and —O— wherein said C₁₋₆ alkyl, carbocyclyl, aryl, heterocyclyl or heteroaryl are optionally further substituted with one, two or three substituents independently selected from the group consisting of halo, —NO₂, C₁₋₆ alkyl, carbocyclyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰; and wherein said C₁₋₆ alkyl, carbocyclyl, aryl, heterocyclyl or heteroaryl are optionally further substituted with one, two or three substituents independently selected from the group consisting of halo, aryl, —NO₂, —CF₃, —N(R²⁰)(R²²), —C(O)—R²⁰, —C(O)—OR²⁰, —C(O)—N(R²⁰)(R²²), —CN, —S(O)₂—R²⁰ and —O—R²⁰;
R¹⁷ is halo, —O—R²⁰ or C₁₋₆ alkyl;
R²⁰ and R²² are in each instance independently selected from the group consisting of hydrogen, C₁₋₆ alkyl, C₂₋₆ alkenyl, C₂₋₆ alkynyl, carbocyclyl, heterocyclyl, aryl and heteroaryl; wherein the C₁₋₆ alkyl, C₂₋₆ alkenyl, C₂₋₆ alkynyl, carbocyclyl, heterocyclyl, aryl and heteroaryl are optionally substituted with one, two or three substituents independently selected from the group consisting of hydroxyl, halo, C₁₋₄ alkyl, acylamino, oxo, —NO₂, —S(O)₂R²⁶, —CN, C₁₋₃ alkoxy, —CF₃, —OCF₃, —OCH₂CF₃, —C(O)—NH₂, aryl, carbocyclyl and heteroaryl; and wherein said heteroaryl is optionally further substituted with C₁₋₄ alkyl or carbocyclyl; or when R²⁰ and R²² are attached to a common nitrogen atom, R²⁰ and R²² may join to form a heterocyclic or heteroaryl ring which is then optionally substituted with one, two or three substituents independently selected from the group consisting of hydroxyl, halo, C₁₋₄ alkyl, aralkyl, aryloxy, aralkyloxy, acylamino, —NO₂, —S(O)₂R²⁶, —CN, C₁₋₄ alkoxy, —CF₃, —OCF₃, aryl, heteroaryl and carbocyclyl; and each R²⁶ is independently selected from the group consisting of hydrogen, C₁₋₄ alkyl, aryl and carbocyclyl; wherein the C₁₋₄ alkyl, aryl and carbocyclyl may be further substituted with from 1 to 3 substituents independently selected from the group consisting of hydroxyl, halo, C₁₋₄ alkoxy, —CF₃ and —OCF₃.

2. A method of treating a pediatric epilepsy, wherein the method comprises administering to a subject in need thereof a compound of Formula (I-a): 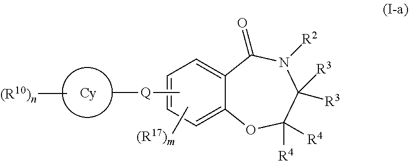
or a pharmaceutically acceptable salt, ester, stereoisomer, mixture of stereoisomers or tautomer thereof, wherein:
Cy is aryl, carbocyclyl, cycloalkenyl, heterocyclyl or heteroaryl;
Q is a covalent bond, —O—C₀₋₂ alkylene, —NR¹¹—C₀₋₂ alkylene, C₂ alkylene, C₂ alkenylene or C₂ alkynylene;
m is 0, 1, 2 or 3;
n is 0, 1, 2, 3, 4 or 5;
each R¹⁰ is independently selected from the group consisting of halo, —NO₂, —CN, —SF₅, —Si(CH₃)₃, —O—R²⁰, —S—R²⁰, —C(O)—R²⁰, —C(O)—OR²⁰, —N(R²⁰)(R²²), —C(O)—N(R²⁰)(R²²), —N(R²⁰)—C(O)—R²², —N(R²⁰)—C(O)—OR²², —N(R²⁰)—S(O)₂—R²⁶, —S(O)₂— R²⁰, —O—S(O)₂—R²⁰, —S(O)₂—N(R²⁰)(R²²), C₁₋₆ alkyl, C₂₋₄ alkenyl, C₂₋₄ alkynyl, carbocyclyl, aryl, heteroaryl and heterocyclyl; and wherein said C₁₋₆ alkyl, C₂₋₄ alkenyl, C₂₋₄ alkynyl, carbocyclyl, aryl, heteroaryl or heterocyclyl are optionally substituted with one, two or three substituents independently selected from the group consisting of halo, —NO₂, aryl, heterocyclyl, heteroaryl, C₁₋₆ alkyl, C₁₋₃ haloalkyl, carbocyclyl, —N(R²⁰)(R²²), —C(O)—R²⁰, —C(O)—OR²⁰, —C(O)—N(R²⁰)(R²²), —CN and —O—R²⁰;
R² is —C₁₋₆ alkylene-R⁵, -L-R⁵, -L-C₁₋₆ alkylene-R⁵, —C₁₋₆ alkylene-L-R⁵ or —C₁₋₆ alkylene-L-C₁₋₆ alkylene-R⁵; wherein each —C₁₋₆ alkyl ene is optionally substituted by one substituent independently selected from the group consisting of C₂₋₄ alkynyl, halo, —NO₂, —CN, —O—R²⁰, —N(R²⁰)(R²²), —C(O)—R²⁰, —C(O)—OR²⁶, —C(O)—N(R²⁰)(R²²), —N(R²⁰)—S(O)₂—R²⁰, carbocyclyl, aryl, heteroaryl or heterocyclyl; and wherein said carbocyclyl, aryl, heteroaryl or heterocyclyl are optionally substituted with one, two or three substituents independently selected from the group consisting of C₁₋₆ alkyl, C₂₋₄ alkynyl, halo, —NO₂, carbocyclyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰;
L is —O—, —S—, —C(O)—, —NHS(O)₂—, —S(O)₂NH—, —C(O)NH— or —NHC(O)—; provided that when R is -L-R or -L-C₁₋₆ alkylene-R, then L is not —O—, —S—, —NHS(O)₂— or —NHC(O)—; and
each R³ is independently hydrogen, deuterium, C₁₋₆ alkyl, carbocyclyl, aryl, heteroaryl or heterocyclyl; wherein said C₁₋₆ alkyl is optionally substituted with one, two or three substituents independently selected from the group consisting of halo, —NO₂, carbocyclyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰; wherein said carbocyclyl, aryl, heterocyclyl or heteroaryl are optionally further substituted with one, two or three substituents independently selected from the group consisting of halo, —NO₂, C₁₋₆ alkyl, aralkyl, carbocyclyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰; and wherein said C₁₋₆ alkyl, aralkyl, carbocyclyl, aryl, heterocyclyl or heteroaryl, are optionally further substituted with one, two or three substituents independently selected from the group consisting of halo, —NO₂, —N(R²⁰)(R²²), —C(O)—R²⁰, —C(O)—OR²⁰, —C(O)—N(R²⁰)(R²²), —CN and —O—R²⁰;
or R² and one of R³ can join together with the atoms to which they are attached to form a heterocyclyl; wherein said heterocyclyl is optionally substituted with one, two or three substituents independently selected from the group consisting of C₁₋₆ alkyl, —O—R²⁰, —N(R²⁰)(R²²), —N(R²⁰)—C(O)—OR²⁰ and —C(O)—OR²⁰; and wherein said C₁₋₆ alkyl is optionally substituted with one, two or three substituents independently selected from the group consisting of halo and heteroaryl;
each R⁴ is independently hydrogen, deuterium, C₁₋₆ alkyl, —C(O)—OR²⁶; —C(O)—N(R²⁶)(R²⁶), carbocyclyl, aryl, heteroaryl or heterocyclyl; wherein said C₁₋₆ alkyl is optionally substituted with one, two or three substituents independently selected from the group consisting of halo, —NO₂, carbocyclyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰; wherein said carbocyclyl, aryl, heterocyclyl or heteroaryl are optionally further substituted with one, two or three substituents independently selected from the group consisting of halo, —NO₂, C₁₋₆ alkyl, aralkyl, carbocyclyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰; and wherein said C₁₋₆ alkyl, aralkyl, carbocyclyl, aryl, heterocyclyl, heteroaryl, are optionally further substituted with one, two or three substituents independently selected from the group consisting of hydroxyl, halo, —NO₂, —N(R²⁰)(R²²), —C(O)—R²⁰, —C(O)—OR²⁰, —C(O)—N(R²⁰)(R²²), —CN and —O—R²⁰;
R⁵ is carbocyclyl, aryl, heteroaryl or heterocyclyl; wherein said carbocyclyl, aryl, heteroaryl or heterocyclyl are optionally substituted with one, two or three substituents independently selected from the group consisting of C₁₋₆ alkyl, C₂₋₄ alkynyl, halo, —NO₂, carbocyclyl, aryl, heterocyclyl, heteroaryl, —N(R²⁰)(R²²), —N(R²⁰)—S(O)₂—R²⁰, —N(R²⁰)—C(O)—R²², —C(O)—R²⁰, —C(O)—OR²⁰, —C(O)—N(R²⁰)(R²²), —CN, oxo and —O— wherein said C₁₋₆ alkyl, carbocyclyl, aryl, heterocyclyl or heteroaryl are optionally further substituted with one, two or three substituents independently selected from the group consisting of halo, —NO₂, C₁₋₆ alkyl, carbocyclyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰; and wherein said C₁₋₆ alkyl, carbocyclyl, aryl, heterocyclyl or heteroaryl are optionally further substituted with one, two or three substituents independently selected from the group consisting of halo, aryl, —NO₂, —CF₃, —N(R²⁰)(R²²), —C(O)—R²⁰, —C(O)—OR²⁰, —C(O)—N(R²⁰)(R²²), —CN, —S(O)₂—R²⁰ and —O—R²⁰;
R¹⁷ is halo, —O—R²⁰ or C₁₋₆ alkyl;
R²⁰ and R²² are in each instance independently selected from the group consisting of hydrogen, C₁₋₆ alkyl, C₂₋₆ alkenyl, C₂₋₆ alkynyl, carbocyclyl, heterocyclyl, aryl and heteroaryl; wherein the C₁₋₆ alkyl, C₂₋₆ alkenyl, C₂₋₆ alkynyl, carbocyclyl, heterocyclyl, aryl and heteroaryl are optionally substituted with one, two or three substituents independently selected from the group consisting of hydroxyl, halo, C₁₋₄ alkyl, acylamino, oxo, —NO₂, —S(O)₂R²⁶, —CN, C₁₋₃ alkoxy, —CF₃, —OCF₃, —OCH₂CF₃, —C(O)—NH₂, aryl, carbocyclyl and heteroaryl; and wherein said heteroaryl is optionally further substituted with C₁₋₄ alkyl or carbocyclyl; or when R²⁰ and R²² are attached to a common nitrogen atom, R²⁰ and R²² may join to form a heterocyclic or heteroaryl ring which is then optionally substituted with one, two or three substituents independently selected from the group consisting of hydroxyl, halo, C₁₋₄ alkyl, aralkyl, aryloxy, aralkyloxy, acylamino, —NO₂, —S(O)₂R²⁶, —CN, C₁₋₄ alkoxy, —CF₃, —OCF₃, aryl, heteroaryl and carbocyclyl; and each R²⁶ is independently selected from the group consisting of hydrogen, C₁₋₄ alkyl, aryl and carbocyclyl; wherein the C₁₋₄ alkyl, aryl and carbocyclyl may be further substituted with from 1 to 3 substituents independently selected from the group consisting of hydroxyl, halo, C₁₋₄ alkoxy, —CF₃ and —OCF₃.

3. A method of treating a refractory epilepsy, wherein the method comprises administering to a subject in need thereof a compound of Formula (I-a): 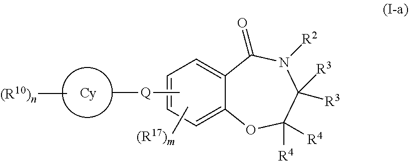
or a pharmaceutically acceptable salt, ester, stereoisomer, mixture of stereoisomers or tautomer thereof, wherein:
Cy is aryl, carbocyclyl, cycloalkenyl, heterocyclyl or heteroaryl;
Q is a covalent bond, —O—C₀₋₂ alkylene, —NR¹¹—C₀₋₂ alkylene, C₂ alkylene, C₂ alkenylene or C₂ alkynylene;
m is 0, 1, 2 or 3;
n is 0, 1, 2, 3, 4 or 5;
each R¹⁰ is independently selected from the group consisting of halo, —NO₂, —CN, —SF₅, —Si(CH₃)₃, —O—R²⁰, —S—R²⁰, —C(O)—R²⁰, —C(O)—OR²⁰, —N(R²⁰)(R²²), —C(O)—N(R²⁰)(R²²), —N(R²⁰)—C(O)—R²², —N(R²⁰)—C(O)—OR²², —N(R²⁰)—S(O)₂—R²⁶, —S(O)₂— R²⁰, —O—S(O)₂—R²⁰, —S(O)₂—N(R²⁰)(R²²), C₁₋₆ alkyl, C₂₋₄ alkenyl, C₂₋₄ alkynyl, carbocyclyl, aryl, heteroaryl and heterocyclyl; and wherein said C₁₋₆ alkyl, C₂₋₄ alkenyl, C₂₋₄ alkynyl, carbocyclyl, aryl, heteroaryl or heterocyclyl are optionally substituted with one, two or three substituents independently selected from the group consisting of halo, —NO₂, aryl, heterocyclyl, heteroaryl, C₁₋₆ alkyl, C₁_₃ haloalkyl, carbocyclyl, —N(R²⁰)(R²²), —C(O)—R²⁰, —C(O)—OR²⁰, —C(O)—N(R²⁰)(R²²), —CN and —O—R²⁰;
R² is —C₁₋₆ alkylene-R⁵, -L-R⁵, -L-C₁₋₆ alkylene-R⁵, —C₁₋₆ alkylene-L-R⁵ or —C₁₋₆ alkylene-L-C₁₋₆ alkylene-R⁵; wherein each —C₁₋₆ alkyl ene is optionally substituted by one substituent independently selected from the group consisting of C₂₋₄ alkynyl, halo, —NO₂, —CN, —O—R²⁰, —N(R²⁰)(R²²), —C(O)—R²⁰, —C(O)—OR²⁶, —C(O)—N(R²⁰)(R²²), —N(R²⁰)—S(O)₂—R²⁰, carbocyclyl, aryl, heteroaryl or heterocyclyl; and wherein said carbocyclyl, aryl, heteroaryl or heterocyclyl are optionally substituted with one, two or three substituents independently selected from the group consisting of C₁₋₆ alkyl, C₂₋₄ alkynyl, halo, —NO₂, carbocyclyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰;
L is —O—, —S—, —C(O)—, —NHS(O)₂—, —S(O)₂NH—, —C(O)NH— or —NHC(O)—; provided that when R is -L-R or -L-C₁₋₆ alkylene-R, then L is not —O—, —S—, —NHS(O)₂— or —NHC(O)—; and
each R³ is independently hydrogen, deuterium, C₁₋₆ alkyl, carbocyclyl, aryl, heteroaryl or heterocyclyl; wherein said C₁₋₆ alkyl is optionally substituted with one, two or three substituents independently selected from the group consisting of halo, —NO₂, carbocyclyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰; wherein said carbocyclyl, aryl, heterocyclyl or heteroaryl are optionally further substituted with one, two or three substituents independently selected from the group consisting of halo, —NO₂, C₁₋₆ alkyl, aralkyl, carbocyclyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰; and wherein said C₁₋₆ alkyl, aralkyl, carbocyclyl, aryl, heterocyclyl or heteroaryl, are optionally further substituted with one, two or three substituents independently selected from the group consisting of halo, —NO₂, —N(R²⁰)(R²²), —C(O)—R²⁰, —C(O)—OR²⁰, —C(O)—N(R²⁰)(R²²), —CN and —O—R²⁰;
or R² and one of R³ can join together with the atoms to which they are attached to form a heterocyclyl; wherein said heterocyclyl is optionally substituted with one, two or three substituents independently selected from the group consisting of C₁₋₆ alkyl, —O—R²⁰, —N(R²⁰)(R²²), —N(R²⁰)—C(O)—OR²⁰ and —C(O)—OR²⁰; and wherein said C₁₋₆ alkyl is optionally substituted with one, two or three substituents independently selected from the group consisting of halo and heteroaryl;
each R⁴ is independently hydrogen, deuterium, C₁₋₆ alkyl, —C(O)—OR²⁶; —C(O)—N(R²⁶)(R²⁶), carbocyclyl, aryl, heteroaryl or heterocyclyl; wherein said C₁₋₆ alkyl is optionally substituted with one, two or three substituents independently selected from the group consisting of halo, —NO₂, carbocyclyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰; wherein said carbocyclyl, aryl, heterocyclyl or heteroaryl are optionally further substituted with one, two or three substituents independently selected from the group consisting of halo, —NO₂, C₁₋₆ alkyl, aralkyl, carbocyclyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰; and wherein said C₁₋₆ alkyl, aralkyl, carbocyclyl, aryl, heterocyclyl, heteroaryl, are optionally further substituted with one, two or three substituents independently selected from the group consisting of hydroxyl, halo, —NO₂, —N(R²⁰)(R²²), —C(O)—R²⁰, —C(O)—OR²⁰, —C(O)—N(R²⁰)(R²²), —CN and —O—R²⁰;
R⁵ is carbocyclyl, aryl, heteroaryl or heterocyclyl; wherein said carbocyclyl, aryl, heteroaryl or heterocyclyl are optionally substituted with one, two or three substituents independently selected from the group consisting of C₁₋₆ alkyl, C₂₋₄ alkynyl, halo, —NO₂, carbocyclyl, aryl, heterocyclyl, heteroaryl, —N(R²⁰)(R²²), —N(R²⁰)—S(O)₂—R²⁰, —N(R²⁰)—C(O)—R²², —C(O)—R²⁰, —C(O)—OR²⁰, —C(O)—N(R²⁰)(R²²), —CN, oxo and —O— wherein said C₁₋₆ alkyl, carbocyclyl, aryl, heterocyclyl or heteroaryl are optionally further substituted with one, two or three substituents independently selected from the group consisting of halo, —NO₂, C₁₋₆ alkyl, carbocyclyl, aryl, heterocyclyl, heteroaryl, —N(R²⁰)(R²²), —C(O)—R²⁰, —C(O)—OR²⁰, —C(O)—N(R²⁰)(R²²), —CN and —O—R²⁰; and wherein said C₁₋₆ alkyl, carbocyclyl, aryl, heterocyclyl or heteroaryl are optionally further substituted with one, two or three substituents independently selected from the group consisting of halo, aryl, —NO₂, —CF₃, —N(R²⁰)(R²²), —C(O)—R²⁰, —C(O)—OR²⁰, —C(O)—N(R²⁰)(R²²), —CN, —S(O)₂—R²⁰ and —O—R²⁰;
R¹⁷ is halo, —O—R²⁰ or C₁₋₆ alkyl;
R²⁰ and R²² are in each instance independently selected from the group consisting of hydrogen, C₁₋₆ alkyl, C₂₋₆ alkenyl, C₂₋₆ alkynyl, carbocyclyl, heterocyclyl, aryl and heteroaryl; wherein the C₁₋₆ alkyl, C₂₋₆ alkenyl, C₂₋₆ alkynyl, carbocyclyl, heterocyclyl, aryl and heteroaryl are optionally substituted with one, two or three substituents independently selected from the group consisting of hydroxyl, halo, C₁₋₄ alkyl, acylamino, oxo, —NO₂, —S(O)₂R²⁶, —CN, C₁₋₃ alkoxy, —CF₃, —OCF₃, —OCH₂CF₃, —C(O)—NH₂, aryl, carbocyclyl and heteroaryl; and wherein said heteroaryl is optionally further substituted with C₁₋₄ alkyl or carbocyclyl; or when R²⁰ and R²² are attached to a common nitrogen atom, R²⁰ and R²² may join to form a heterocyclic or heteroaryl ring which is then optionally substituted with one, two or three substituents independently selected from the group consisting of hydroxyl, halo, C₁₋₄ alkyl, aralkyl, aryloxy, aralkyloxy, acylamino, —NO₂, —S(O)₂R²⁶, —CN, C₁₋₄ alkoxy, —CF₃, —OCF₃, aryl, heteroaryl and carbocyclyl; and each R²⁶ is independently selected from the group consisting of hydrogen, C₁₋₄ alkyl, aryl and carbocyclyl; wherein the C₁₋₄ alkyl, aryl and carbocyclyl may be further substituted with from 1 to 3 substituents independently selected from the group consisting of hydroxyl, halo, C₁₋₄ alkoxy, —CF₃ and —OCF₃.

6. The method of any one of the preceding claims, wherein Q is a covalent bond.

7. The method of any one of the preceding claims, wherein Cy is aryl (e.g., phenyl).

8. The method of any one of the preceding claims, wherein R¹⁰ is —O—R²⁰ (e.g., —OCF₃).

9. The method of any one of the preceding claims, wherein n is 1.

10. The method of any one of the preceding claims, wherein m is 0.

11. The method of any one of the preceding claims, wherein R² is —C₁₋₆ alkylene-R⁵ (e.g., —CH₂—R⁵).

12. The method of any one of the preceding claims, wherein R⁵ is heteroaryl (e.g., a nitrogen-containing heteroaryl, e.g., pyrimidinyl).

13. The method of any one of the preceding claims, wherein each R³ is independently hydrogen.

14. The method of any one of the preceding claims, wherein each R⁴ is independently hydrogen.

15. The method of any one of the preceding claims, wherein the compound of Formula (I-a) is a compound of Formula (I-b): 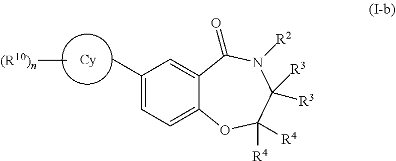
or a pharmaceutically acceptable salt, ester, stereoisomer, mixture of stereoisomers or tautomer thereof, wherein Cy, R², R³, R⁴, R¹⁰, and n are defined as in Formula (I-a).

16. The method of any one of the preceding claims, wherein the compound of Formula (I-a) or (I-b) is selected from a compound disclosed herein.

17. The method of any one of the preceding claims, wherein the compound of Formula (I-a) or (I-b) is: 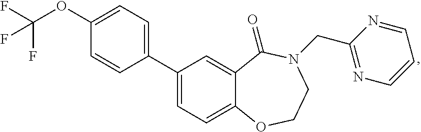 [SMILES: O=C1c2cc(-c3ccc(OC(F)(F)F)cc3)ccc2OCCN1Cc1ncccn1]
or a pharmaceutically acceptable salt, ester, stereoisomer, mixture of stereoisomers or tautomer thereof.

18. The method of any one of the preceding claims, wherein the neurodevelopmental disorder, pediatric epilepsy, or refractory epilepsy comprises Dravet syndrome or a genetic epilepsy.

19. The method of any one of preceding claims, wherein the neurodevelopmental disorder, pediatric epilepsy, or refractory epilepsy is associated with a mutation in ALDH7A1, ALG13, ARHGEF9, ARX, ASAH1, CDKL5, CHD2, CHRNA2, CHRNA4, CHRNB2, CLN8, CNTNAP2, CPA6, CSTB, DEPDC5, DNM1, EEF1A2, EPM2A, EPM2B, GABRA1, GABRB3, GABRG2, GNAO1, GOSR2, GRIN1, GRIN2A, GRIN2B, HCN1, IER3IP1, KCNA2, KCNB1, KCNC1, KCNMA1, KCNQ2, KCNQ3, KCNT1, KCTD7, LGI1, MEF2C, NHLRC1, PCDH19, PLCB1, PNKP, PNPO, PRICKLE1, PRICKLE2, PRRT2, RELN, SCARB2, SCN1A, SCN1B, SCN2A, SCN8A, SCN9A, SIAT9, SIK1, SLC13A5, SLC25A22, SLC2A1, SLC35A2, SLC6A1, SNIP1, SPTAN1, SRPX2, ST3GAL3, STRADA, STX1B, STXBP1, SYN1, SYNGAP1, SZT2, TBC1D24, or WWOX.

20. The method of any one of preceding claims, wherein the neurodevelopmental disorder, pediatric epilepsy, or refractory epilepsy is associated with a mutation in SCN2A.

21. The method of any one of preceding claims, wherein the neurodevelopmental disorder, pediatric epilepsy, or refractory epilepsy is associated with a mutation in SCN8A.

22. The method of any one of preceding claims, wherein the method further comprises administering the compound of formula I-a or I-b to a subject identified as having a mutation in SCN2A.

23. The method of any one of preceding claims, wherein the method further comprises administering the compound of formula I-a or I-b to a subject identified as having a mutation in SCN8A.

24. The method of any one of preceding claims, wherein the method further comprises assaying a sample from a subject to evaluate for the presence of a mutation in SCN2A.

25. The method of any one of preceding claims, wherein the method further comprises assaying a sample from a subject to evaluate for the presence of a mutation in SCN8A.

26. A method for treating a subject having an epilepsy, wherein the epilepsy is characterized by a mutation in an epilepsy associated gene or autism associated gene, the method comprising administering to the subject a modulator (e.g., an inhibitor) of a sodium ion channel to treat the epilepsy.

27. A method of diagnosing and treating a subject suffering from a epilepsy, the method comprising:
a. diagnosing whether the subject has an epilepsy that is susceptible to a treatment, the treatment comprising administration of a modulator (e.g., an inhibitor) of sodium channel, based on the presence of a mutation in an epilepsy associated gene or autism associated gene previously determined to be present in a sample from the subject; and
b. administering to the subject the treatment in an amount effective to treat the epilepsy.

28. The method of claim 26 or 27, wherein the modulator of the sodium channel is eleclazine.

29. The method of any one of claims 26-28, wherein the mutation is a mutation in the gene selected from ALDH7A1, ALG13, ARHGEF9, ARX, ASAH1, CDKL5, CHD2, CHRNA2, CHRNA4, CHRNB2, CLN8, CNTNAP2, CPA6, CSTB, DEPDC5, DNM1, EEF1A2, EPM2A, EPM2B, GABRA1, GABRB3, GABRG2, GNAO1, GOSR2, GRIN1, GRIN2A, GRIN2B, HCN1, IER3IP1, KCNA2, KCNB1, KCNC1, KCNMA1, KCNQ2, KCNQ3, KCNT1, KCTD7, LGI1, MEF2C, NHLRC1, PCDH19, PLCB1, PNKP, PNPO, PRICKLE1, PRICKLE2, PRRT2, RELN, SCARB2, SCN1A, SCN1B, SCN2A, SCN8A, SCN9A, SIAT9, SIK1, SLC13A5, SLC25A22, SLC2A1, SLC35A2, SLC6A1, SNIP1, SPTAN1, SRPX2, ST3GAL3, STRADA, STX1B, STXBP1, SYN1, SYNGAP1, SZT2, TBC1D24, and WWOX.

30. The method of any one of claims 26-29, wherein the mutation is a mutation in SCN2A.

31. The method of any one of claims 26-30, wherein the mutation is a mutation in SCN8A.

32. The method of any one of claims 26-31, wherein the method further comprises administering a modulator (e.g., an inhibitor) of a sodium ion to a subject identified as having a mutation in SCN2A.

33. The method of any one of claims 26-32, wherein the method further comprises administering a modulator (e.g., an inhibitor) of a sodium ion to a subject identified as having a mutation in SCN8A.

34. The method of any one of claims 26-33, wherein the method further comprises assaying a sample from a subject to evaluate for the presence of a mutation in SCN2A.

35. The method of any one of claims 26-34, wherein the method further comprises assaying a sample from a subject to evaluate for the presence of a mutation in SCN8A.

36. The method of any one of claims 26-35, wherein the mutation is a spontaneous mutation.

37. The method of any one of claims 26-36, wherein the mutation is determined in the subject by sequencing of the DNA or RNA from a sample of the subject.

38. The method of any one of claims 26-37, wherein the sequencing comprises sequencing of the whole genome or whole exome of the subject.

39. The method of any one of claims 26-38, wherein the sequencing comprises sequencing a panel of genes from the subject.

40. The method of any one of claims 26-39, wherein the mutation is a mutation in an epilepsy associated gene.

41. The method of any one of claims 26-40, wherein the mutation is a mutation identified in the Epilepsy phenome/Genome project.

42. The method of any one of claims 26-41, wherein the mutation is in an autism associated gene.

43. The method of any one of claims 26-42, wherein the inhibitor is an inhibitor of late sodium current (INaL).

44. A method for treating a subject having an epilepsy, wherein the subject is characterized as having normal cardiac function, the method comprising administering to the subject a modulator (e.g., an inhibitor) of a sodium ion channel to treat the epilepsy.

45. The method of claim 44, wherein the modulator is a modulator (e.g., an inhibitor) of late sodium current (INaL).

46. The method of claim 44 or 45, wherein the subject is characterized as being without structural heart disease.

47. The method of any one of claims 44-46, wherein the subject is characterized as being without arrhythmias.

48. The method of any one of claims 44-47, further comprising receiving information characterizing the subject based on an electrocardiogram.

49. The method of any one of claims 44-48, further comprising receiving information characterizing the subject based on a cardiac ultrasound.

50. The method of any one of claims 44-49, further comprising receiving information characterizing the subject based on an echocardiogram (e.g., relating to structural heart disease or arrhythmia).

51. The method of any one of claims 44-50, further comprising receiving information characterizing the subject based on a cardiac MRI.

52. The method of any one of claims 44-51, further comprising receiving information characterizing the subject based on a cardiac CT.
